(19) United States
(12) Patent Application Publication
(10) Pub. No.: US 2024/0050562 A1
(43) Pub. Date: Feb. 15, 2024

(54) INHIBITOR OF CELL PROLIFERATION IN OBINUTUZUMAB RESISTANT CD20-POSITIVE CANCER, AND MEDICINAL COMPOSITION, MEDICINE, PRODUCTION, METHOD FOR INHIBITING CELL PROLIFERATION, THERAPEUTIC METHOD, TYPE II ANTI-CD20 ANTIBODY, COMPOUNDS, COMBINATION OF SAME, ENHANCER AND INDUCER, EACH RELATING THERETO

(71) Applicant: Chugai Seiyaku Kabushiki Kaisha, Tokyo (JP)

(72) Inventors: Takaaki FUJIMURA, Kanagawa (JP); Yoriko KASHIMA, Kanagawa (JP); Natsumi OKA, Kanagawa (JP)

(21) Appl. No.: 17/766,280

(22) PCT Filed: Sep. 18, 2020
(86) PCT No.: PCT/JP2020/035457

§ 371 Date: Apr. 4, 2022

(30) Foreign Application Priority Data
Oct. 4, 2019 (JP) 2019-184149

(51) Int. Cl. A61K 39/395; A61K 31/573; A61K 31/704; A61K 31/475; A61P 35/00

(52) U.S. Cl. CPC A61K 39/3955; A61K 31/573; A61K 31/704; A61K 31/475; A61P 35/00

(57) ABSTRACT

There are cases of CD20-positive cancer where the cancer is tolerant to obinutuzumab and cases where the cancer recurs after an obinutuzumab-containing treatment.

For CD20-positive cancer that is tolerant to obinutuzumab or CD20-positive cancer that has recurred following an obinutuzumab-containing treatment, a type II anti-CD20 antibody is used in combination with one or more compounds selected from the group consisting of prednisolone, doxorubicin, vincristine, and salts and prodrugs thereof. Among these compounds, prednisolone, doxorubicin, and salts and prodrugs thereof may be selected. Furthermore, among these compounds, prednisolone or a salt or prodrug thereof may be selected.

### TECHNICAL FIELD

The present invention relates to agents for suppressing cell proliferation of obinutuzumab-tolerant CD20-positive cancer, as well as pharmaceutical compositions, medicaments, manufacture, methods for suppressing cell proliferation, treatment methods, type II anti-CD20 antibodies, compounds, combinations thereof, and enhancing agents and inducing agents, pertaining to the same.

### BACKGROUND ART

Patent Document 1 discloses the combined use of a type II anti-CD20 antibody having increased antibody-dependent cellular cytotoxicity (ADCC) and one or more chemotherapeutic agents selected from the group consisting of cyclophosphamide, vincristine, and doxorubicin in the treatment of a CD20-positive cancer.

Patent Document 1: WO 2009/118142 A

### SUMMARY OF INVENTION

There are cases of CD20-positive cancer where the cancer is tolerant to obinutuzumab and cases where the cancer recurs after an obinutuzumab-containing treatment.

The present inventors examined means for increasing the effects of treatments using type II anti-CD20 antibodies, especially obinutuzumab, on CD20-positive cancer that is tolerant to obinutuzumab or CD20-positive cancer that has recurred following an obinutuzumab-containing treatment.

The present inventors found that with the combined use of one or more compounds selected from the group consisting of prednisolone, doxorubicin, vincristine, and salts and prodrugs thereof, the effects of treatments using type II anti-CD20 antibodies, especially obinutuzumab, are increased against CD20-positive cancer that is tolerant to obinutuzumab or CD20-positive cancer that has recurred following an obinutuzumab-containing treatment. Among these compounds, prednisolone, doxorubicin, and salts and prodrugs thereof may be selected. Furthermore, among these compounds, prednisolone or a salt or prodrug thereof may be selected.

The following invention is provided on the basis of these teachings.
(1) An agent for suppressing cell proliferation of an obinutuzumab-tolerant CD20-positive cancer, wherein the agent comprises a type II anti-CD20 antibody and is used in combination with chemotherapy comprising administration of one or more compounds selected from the group consisting of prednisolone, doxorubicin, vincristine, and salts and prodrugs thereof.
(2) An agent for suppressing cell proliferation of an obinutuzumab-tolerant CD20-positive cancer by combined use with chemotherapy comprising administration of one or more compounds selected from the group consisting of prednisolone, doxorubicin, vincristine, and salts and prodrugs thereof, wherein the agent comprises a type II anti-CD20 antibody.
(3) An agent for suppressing cell proliferation of an obinutuzumab-tolerant CD20-positive cancer, wherein the agent comprises one or more compounds selected from the group consisting of prednisolone, doxorubicin, vincristine, and salts and prodrugs thereof, and is used in combination with treatment with a type II anti-CD20 antibody.
(4) An agent for suppressing cell proliferation of an obinutuzumab-tolerant CD20-positive cancer by combined use with treatment with a type II anti-CD20 antibody, wherein the agent comprises one or more compounds selected from the group consisting of prednisolone, doxorubicin, vincristine, and salts and prodrugs thereof.
(5) A medicament for suppressing cell proliferation of an obinutuzumab-tolerant CD20-positive cancer, wherein a type II anti-CD20 antibody and one or more compounds selected from the group consisting of prednisolone, doxorubicin, vincristine, and salts and prodrugs thereof are concurrently, separately, or sequentially administered in combination.
(6) The agent of any one of (1)-(4), wherein the CD20-positive cancer is B-cell non-Hodgkin's lymphoma.
(7) The medicament of (5), wherein the CD20-positive cancer is B-cell non-Hodgkin's lymphoma.
(8) The agent of any one of (1)-(4) and (6), wherein the type II anti-CD20 antibody is obinutuzumab.
(9) The medicament of (5) or (7), wherein the type II anti-CD20 antibody is obinutuzumab.
(10) The agent of any one of (1)-(4), (6), and (8), wherein the one or more compounds are selected from the group consisting of prednisolone, doxorubicin, and salts and prodrugs thereof.
(11) The medicament of any one of (5), (7), and (9), wherein the one or more compounds are selected from the group consisting of prednisolone, doxorubicin, and salts and prodrugs thereof.
(12) The agent of any one of (1)-(4), (6), (8), and (10), wherein the obinutuzumab-tolerant CD20-positive cancer is a CD20-positive cancer which was previously treated with obinutuzumab.
(13) The medicament of any one of (5), (7), (9), and (11), wherein the obinutuzumab-tolerant CD20-positive cancer is a CD20-positive cancer which was previously treated with obinutuzumab.
(14) The agent of any one of (1)-(4), (6), (8), (10), and (12), wherein the obinutuzumab-tolerant CD20-positive cancer is a cancer which has recurred after initiation of maintenance therapy in which obinutuzumab is administered alone after induction therapy using obinutuzumab.
(15) The medicament of any one of (5), (7), (9), (11), and (13), wherein the obinutuzumab-tolerant CD20-positive cancer is a cancer which has recurred after initiation of maintenance therapy in which obinutuzumab is administered alone after induction therapy using obinutuzumab.
(16) A pharmaceutical composition for treating an obinutuzumab-tolerant CD20-positive cancer, wherein the pharmaceutical composition comprises a type II anti-CD20 antibody and is used in combination with chemotherapy comprising administration of one or more compounds selected from the group consisting of prednisolone, doxorubicin, vincristine, and salts and prodrugs thereof.
(17) A pharmaceutical composition for treating an obinutuzumab-tolerant CD20-positive cancer by combined use with chemotherapy comprising administration of one or more compounds selected from the group consisting of prednisolone, doxorubicin, vincristine, and salts and prodrugs thereof, wherein the pharmaceutical composition comprises a type II anti-CD20 antibody.
(18) A pharmaceutical composition for treating an obinutuzumab-tolerant CD20-positive cancer, wherein the pharmaceutical composition comprises one or more compounds selected from the group consisting of prednisolone, doxorubicin, vincristine, and salts and prodrugs thereof, and is used in combination with treatment with a type II anti-CD20 antibody.
(19) A pharmaceutical composition for treating an obinutuzumab-tolerant CD20-positive cancer by combined use with treatment with a type II anti-CD20 antibody, wherein the pharmaceutical composition comprises one or more compounds selected from the group consisting of prednisolone, doxorubicin, vincristine, and salts and prodrugs thereof.
(20) A pharmaceutical composition for treating an obinutuzumab-tolerant CD20-positive cancer, wherein a type II anti-CD20 antibody and one or more compounds selected from the group consisting of prednisolone, doxorubicin, vincristine, and salts and prodrugs thereof are concurrently, separately, or sequentially administered in combination.
(21) The pharmaceutical composition of any one of (16)-(20), wherein the CD20-positive cancer is B-cell non-Hodgkin's lymphoma.
(22) The pharmaceutical composition of any one of (16)-(21), wherein the type II anti-CD20 antibody is obinutuzumab.
(23) The pharmaceutical composition of any one of (16)-(22), wherein the one or more compounds are selected from the group consisting of prednisolone, doxorubicin, and salts and prodrugs thereof.
(24) The pharmaceutical composition of any one of (16)-(23), wherein the obinutuzumab-tolerant CD20-positive cancer is a CD20-positive cancer which was previously treated with obinutuzumab.
(25) The pharmaceutical composition of any one of (16)-(24), wherein the obinutuzumab-tolerant CD20-positive cancer is a cancer which has recurred after initiation of maintenance therapy in which obinutuzumab is administered alone after induction therapy using obinutuzumab.
(26) Use of a type II anti-CD20 antibody in the manufacture of an agent for suppressing cell proliferation of an obinutuzumab-tolerant CD20-positive cancer by combined use with chemotherapy comprising administration of one or more compounds selected from the group consisting of prednisolone, doxorubicin, vincristine, and salts and prodrugs thereof.
(27) Use of one or more compounds selected from the group consisting of prednisolone, doxorubicin, vincristine, and salts and prodrugs thereof, in the manufacture of an agent for suppressing cell proliferation of an obinutuzumab-tolerant CD20-positive cancer by combined use with treatment with a type II anti-CD20 antibody.
(28) Use of a type II anti-CD20 antibody in the manufacture of a pharmaceutical composition for treating an obinutuzumab-tolerant CD20-positive cancer by combined use with chemotherapy comprising administration of one or more compounds selected from the group consisting of prednisolone, doxorubicin, vincristine, and salts and prodrugs thereof.
(29) Use of one or more compounds selected from the group consisting of prednisolone, doxorubicin, vincristine, and salts and prodrugs thereof, in the manufacture of a pharmaceutical composition for treating an obinutuzumab-tolerant CD20-positive cancer by combined use with treatment with a type II anti-CD20 antibody.
(30) Use of a type II anti-CD20 antibody and one or more compounds selected from the group consisting of prednisolone, doxorubicin, vincristine, and salts and prodrugs thereof, in the manufacture of a pharmaceutical composition for treating an obinutuzumab-tolerant CD20-positive cancer.
(31) The use of any one of (26)-(30), wherein the CD20-positive cancer is B-cell non-Hodgkin's lymphoma.
(32) The use of any one of (26)-(31), wherein the type II anti-CD20 antibody is
(33) The use of any one of (26)-(32), wherein the one or more compounds are selected from the group consisting of prednisolone, doxorubicin, and salts and prodrugs thereof.
(34) The use of any one of (26)-(33), wherein the obinutuzumab-tolerant CD20-positive cancer is a CD20-positive cancer which was previously treated with obinutuzumab.
(35) The use of any one of (26)-(34), wherein the obinutuzumab-tolerant CD20-positive cancer is a cancer which has recurred after initiation of maintenance therapy in which obinutuzumab is administered alone after induction therapy using obinutuzumab.
(36) A type II anti-CD20 antibody for use in the suppression of cell proliferation of an obinutuzumab-tolerant CD20-positive cancer by combined use with chemotherapy comprising administration of one or more compounds selected from the group consisting of prednisolone, doxorubicin, vincristine, and salts and prodrugs thereof.
(37) One or more compounds selected from the group consisting of prednisolone, doxorubicin, vincristine, and salts and prodrugs thereof, for use in the suppression of cell proliferation of an obinutuzumab-tolerant CD20-positive cancer by combined use with treatment with a type II anti-CD20 antibody.
(38) A combination of a type II anti-CD20 antibody and one or more compounds selected from the group consisting of prednisolone, doxorubicin, vincristine, and salts and prodrugs thereof, for use in the suppression of cell proliferation of an obinutuzumab-tolerant CD20-positive cancer.
(39) A type II anti-CD20 antibody for use in the treatment of an obinutuzumab-tolerant CD20-positive cancer by combined use with chemotherapy comprising administration of one or more compounds selected from the group consisting of prednisolone, doxorubicin, vincristine, and salts and prodrugs thereof.
(40) One or more compounds selected from the group consisting of prednisolone, doxorubicin, vincristine, and salts and prodrugs thereof, for use in the treatment of an obinutuzumab-tolerant CD20-positive cancer by combined use with treatment with a type II anti-CD20 antibody.
(41) A combination of a type II anti-CD20 antibody and one or more compounds selected from the group consisting of prednisolone, doxorubicin, vincristine, and salts and prodrugs thereof, or a combination thereof, for use in the treatment of an obinutuzumab-tolerant CD20-positive cancer.
(42) The type II anti-CD20 antibody of (36) or (39), wherein the CD20-positive cancer is B-cell non-Hodgkin's lymphoma.
(43) The one or more compounds of (37) or (40), wherein the CD20-positive cancer is B-cell non-Hodgkin's lymphoma.
(44) The combination of (38) or (41), wherein the CD20-positive cancer is B-cell non-Hodgkin's lymphoma.
(45) The type II anti-CD20 antibody of any one of (36), (39), and (42), wherein the type II anti-CD20 antibody is obinutuzumab.
(46) The one or more compounds of any one of (37), (40), and (43), wherein the type II anti-CD20 antibody is obinutuzumab.
(47) The combination of any one of (38), (41), and (44), wherein the type II anti-CD20 antibody is obinutuzumab.
(48) The type II anti-CD20 antibody of any one of (36), (39), (42), and (45), wherein the one or more compounds are selected from the group consisting of prednisolone, doxorubicin, and salts and prodrugs thereof.
(49) The one or more compounds of any one of (37), (40), (43), and (46), wherein the one or more compounds are selected from the group consisting of prednisolone, doxorubicin, and salts and prodrugs thereof.
(50) The combination of any one of (38), (41), (44), and (47), wherein the one or more compounds are selected from the group consisting of prednisolone, doxorubicin, and salts and prodrugs thereof.
(51) The type II anti-CD20 antibody of any one of (36), (39), (42), (45), and (48), wherein the obinutuzumab-tolerant CD20-positive cancer is a CD20-positive cancer which was previously treated with obinutuzumab.
(52) The one or more compounds of any one of (37), (40), (43), (46), and (49), wherein the obinutuzumab-tolerant CD20-positive cancer is a CD20-positive cancer which was previously treated with obinutuzumab.
(53) The combination of any one of (38), (41), (44), (47), and (50), wherein the obinutuzumab-tolerant CD20-positive cancer is a CD20-positive cancer which was previously treated with obinutuzumab.
(54) The type II anti-CD20 antibody of any one of (36), (39), (42), (45), (48), and (51), wherein the obinutuzumab-tolerant CD20-positive cancer is a cancer which has recurred after initiation of maintenance therapy in which obinutuzumab is administered alone after induction therapy using obinutuzumab.
(55) The one or more compounds of any one of (37), (40), (43), (46), (49), and (52), wherein the obinutuzumab-tolerant CD20-positive cancer is a cancer which has recurred after initiation of maintenance therapy in which obinutuzumab is administered alone after induction therapy using obinutuzumab.
(56) A method for suppressing cell proliferation of an obinutuzumab-tolerant CD20-positive cancer, the method comprising:
   i) administering a type II anti-CD20 antibody; and
   ii) administering one or more compounds selected from the group consisting of prednisolone, doxorubicin, vincristine, and salts and prodrugs thereof;
   to an obinutuzumab-tolerant CD20-positive cancer cell.
(57) The method of (56), wherein the CD20-positive cancer is B-cell non-Hodgkin's lymphoma.
(58) The method of (56) or (57), wherein the type II anti-CD20 antibody is obinutuzumab.
(59) The method of any one of (56)-(58), wherein the one or more compounds are selected from the group consisting of prednisolone, doxorubicin, and salts and prodrugs thereof.
(60) The method of any one of (56)-(59), wherein the obinutuzumab-tolerant CD20-positive cancer is a CD20-positive cancer which was previously treated with obinutuzumab.
(61) The method of any one of (56)-(60), wherein the obinutuzumab-tolerant CD20-positive cancer is a cancer which has recurred after initiation of maintenance therapy in which obinutuzumab is administered alone after induction therapy using
(62) The method of any one of (56)-(61), wherein the method is an in vitro method or a non-human in vivo method.
(63) A method for treating an obinutuzumab-tolerant CD20-positive cancer, the method comprising:
   i) administering a type II anti-CD20 antibody; and
   ii) administering one or more compounds selected from the group consisting of prednisolone, doxorubicin, vincristine, and salts and prodrugs thereof;
   to an organism having an obinutuzumab-tolerant CD20-positive cancer.
(64) The treatment method of (63), wherein the CD20-positive cancer is B-cell non-Hodgkin's lymphoma.
(65) The treatment method of (63) or (64), wherein the type II anti-CD20 antibody is obinutuzumab.
(66) The treatment method of any one of (63)-(65), wherein the one or more compounds are selected from the group consisting of prednisolone, doxorubicin, and salts and prodrugs thereof.
(67) The treatment method of any one of (63)-(66), wherein the obinutuzumab-tolerant CD20-positive cancer is a CD20-positive cancer which was previously treated with obinutuzumab.
(68) The treatment method of any one of (63)-(67), wherein the obinutuzumab-tolerant CD20-positive cancer is a cancer which has recurred after initiation of maintenance therapy in which obinutuzumab is administered alone after induction therapy using obinutuzumab.
(69) An enhancing agent of cell cycle arrest or cell death by a type II anti-CD20 antibody in an obinutuzumab-tolerant CD20-positive cancer cell, wherein the enhancing agent comprises prednisolone or a salt or prodrug thereof.
(70) An enhancing agent for enhancing cell cycle arrest or cell death against an obinutuzumab-tolerant CD20-positive cancer cell, wherein the enhancing agent comprises prednisolone or a salt or prodrug thereof, and is administered by combined use with a type II anti-CD20 antibody, wherein the combined use enhances cell cycle arrest or cell death against an obinutuzumab-tolerant CD20-positive cancer cell.
(71) An enhancing agent for enhancing induction of cell cycle arrest or cell death against an obinutuzumab-tolerant CD20-positive cancer cell, wherein the enhancing agent comprises prednisolone or a salt or prodrug thereof, and is used in combination with a type II anti-CD20 antibody.
(72) An inducing agent of cell cycle arrest or cell death in an obinutuzumab-tolerant CD20-positive cancer cell, for enhancing induction of cell cycle arrest or cell death against an obinutuzumab-tolerant CD20-positive cancer cell, wherein the inducing agent comprises a type II anti-CD20 antibody and is administered by combined use with chemotherapy comprising administration of prednisolone or a salt or prodrug thereof, and the combined use enhances induction of cell cycle arrest or cell death against the obinutuzumab-tolerant CD20-positive cancer cell.
(73) An inducing agent for enhancing induction of cell cycle arrest or cell death against an obinutuzumab-tolerant CD20-positive cancer cell, wherein the inducing agent comprises a type II anti-CD20 antibody and is used in combination with chemotherapy comprising administration of prednisolone or a salt or prodrug thereof.
(74) An agent for enhancing induction of cell cycle arrest or cell death against an obinutuzumab-tolerant CD20-positive cancer cell, wherein a type II anti-CD20 antibody and prednisolone or a salt or prodrug thereof are concurrently, separately, or sequentially administered in combination.
(75) The agent of any one of (69)-(74), wherein the prodrug is prednisone, and the type II anti-CD20 antibody and the prednisone are administered to an organism.
(76) The agent of any one of (69)-(75), wherein the cell cycle arrest is an arrest in G₀/G₁ phase.
(77) The agent of any one of (69)-(76), wherein the CD20-positive cancer is B-cell non-Hodgkin's lymphoma.
(78) The agent of any one of (69)-(77), wherein the type II anti-CD20 antibody is obinutuzumab.
(79) The agent of any one of (69)-(78), wherein the obinutuzumab-tolerant CD20-positive cancer is a CD20-positive cancer which was previously treated with obinutuzumab.
(80) The agent of any one of (69)-(79), wherein the obinutuzumab-tolerant CD20-positive cancer is a cancer which has recurred after initiation of maintenance therapy in which obinutuzumab is administered alone after induction therapy using
(81) Use of prednisolone or a salt or prodrug thereof, in the manufacture of an enhancing agent of cell cycle arrest or cell death by a type II anti-CD20 antibody in an obinutuzumab-tolerant CD20-positive cancer cell.
(82) Use of a type II anti-CD20 antibody in the manufacture of an inducing agent of cell cycle arrest or cell death in an obinutuzumab-tolerant CD20-positive cancer cell, for enhancing induction of cell cycle arrest or cell death against an obinutuzumab-tolerant CD20-positive cancer cell, wherein the inducing agent is administered by combined use with chemotherapy comprising administration of prednisolone or a salt or prodrug thereof, and the combined use enhances induction of cell cycle arrest or cell death against the obinutuzumab-tolerant CD20-positive cancer cell.
(83) Use of a type II anti-CD20 antibody and prednisolone or a salt or prodrug thereof, in the manufacture of an agent for enhancing induction of cell cycle arrest or cell death in an obinutuzumab-tolerant CD20-positive cancer cell.
(84) The use of any one of (81)-(83), wherein the prodrug is prednisone, and the type II anti-CD20 antibody and the prednisone are administered to an organism.
(85) The use of any one of (81)-(84), wherein the cell cycle arrest is an arrest in G₀/G₁ phase.
(86) The use of any one of (81)-(85), wherein the CD20-positive cancer is B-cell non-Hodgkin's lymphoma.
(87) The use of any one of (81)-(86), wherein the type II anti-CD20 antibody is obinutuzumab.
(88) The use of any one of (81)-(87), wherein the obinutuzumab-tolerant CD20-positive cancer is a CD20-positive cancer which was previously treated with obinutuzumab.
(89) The use of any one of (81)-(88), wherein the obinutuzumab-tolerant CD20-positive cancer is a cancer which has recurred after initiation of maintenance therapy in which obinutuzumab is administered alone after induction therapy using
(90) Prednisolone or a salt or prodrug thereof, for use in the enhancement of cell cycle arrest or cell death by a type II anti-CD20 antibody in an obinutuzumab-tolerant CD20-positive cancer cell.
(91) A type II anti-CD20 antibody, for use in the enhancement of induction of cell cycle arrest or cell death against an obinutuzumab-tolerant CD20-positive cancer cell, by combined use with chemotherapy comprising administration of prednisolone or a salt or prodrug thereof.
(92) A combination of a type II anti-CD20 antibody and prednisolone or a salt or prodrug thereof, for use in the enhancement of induction of cell cycle arrest or cell death against an obinutuzumab-tolerant CD20-positive cancer cell.
(93) The prednisolone or a salt or prodrug thereof of (90), wherein the prodrug is prednisone, and the type II anti-CD20 antibody and the prednisone are administered to an organism.
(94) The type II anti-CD20 antibody of (91), wherein the prodrug is prednisone, and the type II anti-CD20 antibody and the prednisone are administered to an organism.
(95) The combination of (92), wherein the prodrug is prednisone, and the type II anti-CD20 antibody and the prednisone are administered to an organism.
(96) The prednisolone or a salt or prodrug thereof of (90) or (93), wherein the cell cycle arrest is an arrest in G₀/G₁ phase.
(97) The type II anti-CD20 antibody of (91) or (94), wherein the cell cycle arrest is an arrest in G₀/G₁ phase.
(98) The combination of (92) or (95), wherein the cell cycle arrest is an arrest in G₀/G₁ phase.
(99) The prednisolone or a salt or prodrug thereof of any one of (90), (93), and (96), wherein the CD20-positive cancer is B-cell non-Hodgkin's lymphoma.
(100) The type II anti-CD20 antibody of any one of (91), (94), and (97), wherein the CD20-positive cancer is B-cell non-Hodgkin's lymphoma.
(101) The combination any one of (92), (95), and (98), wherein the CD20-positive cancer is B-cell non-Hodgkin's lymphoma.
(102) The prednisolone or a salt or prodrug thereof of any one of (90), (93), (96), and (99), wherein the type II anti-CD20 antibody is obinutuzumab.
(103) The type II anti-CD20 antibody of any one of (91), (94), (97), and (100), wherein the type II anti-CD20 antibody is obinutuzumab.
(104) The combination of any one of (92), (95), (98), and (101), wherein the type II anti-CD20 antibody is obinutuzumab.
(105) The prednisolone or a salt or prodrug thereof of any one of (90), (93), (96), (99), and (102), wherein the obinutuzumab-tolerant CD20-positive cancer is a CD20-positive cancer which was previously treated with obinutuzumab.
(106) The type II anti-CD20 antibody of any one of (91), (94), (97), (100), and (103), wherein the obinutuzumab-tolerant CD20-positive cancer is a CD20-positive cancer which was previously treated with obinutuzumab.
(107) The combination of any one of (92), (95), (98), (101), and (104), wherein the obinutuzumab-tolerant CD20-positive cancer is a CD20-positive cancer which was previously treated with obinutuzumab.
(108) The prednisolone or a salt or prodrug thereof of any one of (90), (93), (96), (99), (102), and (105), wherein the obinutuzumab-tolerant CD20-positive cancer is a cancer which has recurred after initiation of maintenance therapy in which obinutuzumab is administered alone after induction therapy using obinutuzumab.
(109) The type II anti-CD20 antibody of any one of (91), (94), (97), (100), (103), and (106), wherein the obinutuzumab-tolerant CD20-positive cancer is a cancer which has recurred after initiation of maintenance therapy in which obinutuzumab is administered alone after induction therapy using obinutuzumab.
(110) The combination of any one of (92), (95), (98), (101), (104), and (107), wherein the obinutuzumab-tolerant CD20-positive cancer is a cancer which has recurred after initiation of maintenance therapy in which obinutuzumab is administered alone after induction therapy using obinutuzumab.
(111) A method of enhancing cell cycle arrest or cell death by a type II anti-CD20 antibody in an obinutuzumab-tolerant CD20-positive cancer cell, the method comprising administering prednisolone or a salt or prodrug thereof.
(112) A method of inducing cell cycle arrest or cell death in an obinutuzumab-tolerant CD20-positive cancer cell, the method comprising administering a type II anti-CD20 antibody by combined use with chemotherapy comprising administration of prednisolone or a salt or prodrug thereof, wherein the combined use enhances cell cycle arrest or cell death against an obinutuzumab-tolerant CD20-positive cancer cell.
(113) The method of (111) or (112), wherein the prodrug is prednisone, and the type II anti-CD20 antibody and the prednisone are administered to an organism.
(114) The method of any one of (111)-(113), wherein the cell cycle arrest is an arrest in G₀/G₁ phase.
(115) The method of any one of (111)-(114), wherein the CD20-positive cancer is B-cell non-Hodgkin's lymphoma.
(116) The method of any one of (111)-(115), wherein the type II anti-CD20 antibody is obinutuzumab.
(117) The method of any one of (111)-(116), wherein the obinutuzumab-tolerant CD20-positive cancer is a CD20-positive cancer which was previously treated with obinutuzumab.
(118) The method of any one of (111)-(117), wherein the obinutuzumab-tolerant CD20-positive cancer is a cancer which has recurred after initiation of maintenance therapy in which obinutuzumab is administered alone after induction therapy using obinutuzumab.
(119) The method of any one of (111)-(118), wherein the method is an in vitro method or a non-human in vivo method.
(120) An agent for suppressing cell proliferation of an obinutuzumab-tolerant CD20-positive cancer, wherein the agent comprises a type II anti-CD20 antibody and is used in combination with chemotherapy comprising administration of a caspase activating agent.
(121) An agent for suppressing cell proliferation of an obinutuzumab-tolerant CD20-positive cancer by combined use with chemotherapy comprising administration of a caspase activating agent, wherein the agent comprises a type II anti-CD20 antibody.
(122) An agent for suppressing cell proliferation of an obinutuzumab-tolerant CD20-positive cancer, wherein the agent comprises a caspase activating agent and is used in combination with treatment with a type II anti-CD20 antibody.
(123) An agent for suppressing cell proliferation of an obinutuzumab-tolerant CD20-positive cancer by combined use with treatment with a type II anti-CD20 antibody, wherein the agent comprises a caspase activating agent.
(124) A medicament for suppressing cell proliferation of an obinutuzumab-tolerant CD20-positive cancer, wherein a type II anti-CD20 antibody and a caspase activating agent are concurrently, separately, or sequentially administered in combination.
(125) The agent of any one of (120)-(123), wherein the CD20-positive cancer is B-cell non-Hodgkin's lymphoma.
(126) The medicament of (124), wherein the CD20-positive cancer is B-cell non-Hodgkin's lymphoma.
(127) The agent of any one of (120)-(123) and (125), wherein the type II anti-CD20 antibody is obinutuzumab.
(128) The medicament of (124) or (126), wherein the type II anti-CD20 antibody is obinutuzumab.
(129) The agent of any one of (120)-(123), (125), and (127), wherein the caspase activating agent is doxorubicin or a salt thereof.
(130) The medicament of any one of (124), (126), and (128), wherein the caspase activating agent is doxorubicin or a salt thereof.
(131) The agent of any one of (120)-(123), (125), (127), and (129), wherein the obinutuzumab-tolerant CD20-positive cancer is a CD20-positive cancer which was previously treated with obinutuzumab.
(132) The medicament of any one of (124), (126), (128), and (130), wherein the obinutuzumab-tolerant CD20-positive cancer is a CD20-positive cancer which was previously treated with obinutuzumab.
(133) The agent of any one of (120)-(123), (125), (127), (129), and (131), wherein the obinutuzumab-tolerant CD20-positive cancer is a cancer which has recurred after initiation of maintenance therapy in which obinutuzumab is administered alone after induction therapy using obinutuzumab.
(134) The medicament of any one of (124), (126), (128), (130), and (132), wherein the obinutuzumab-tolerant CD20-positive cancer is a cancer which has recurred after initiation of maintenance therapy in which obinutuzumab is administered alone after induction therapy using obinutuzumab.
(135) A pharmaceutical composition for treating an obinutuzumab-tolerant CD20-positive cancer, wherein the pharmaceutical composition comprises a type II anti-CD20 antibody and is used in combination with chemotherapy comprising administration of a caspase activating agent.
(136) A pharmaceutical composition for treating an obinutuzumab-tolerant CD20-positive cancer by combined use with chemotherapy comprising administration of a caspase activating agent, wherein the pharmaceutical composition comprises a type II anti-CD20 antibody.
(137) A pharmaceutical composition for treating an obinutuzumab-tolerant CD20-positive cancer, wherein the pharmaceutical composition comprises a caspase activating agent and is used in combination with treatment with a type II anti-CD20 antibody.
(138) A pharmaceutical composition for treating an obinutuzumab-tolerant CD20-positive cancer by combined use with treatment with a type II anti-CD20 antibody, wherein the pharmaceutical composition comprises a caspase activating agent.
(139) A pharmaceutical composition for treating an obinutuzumab-tolerant CD20-positive cancer, wherein a type II anti-CD20 antibody and a caspase activating agent are concurrently, separately, or sequentially administered in combination.
(140) The pharmaceutical composition of any one of (135)-(139), wherein the CD20-positive cancer is B-cell non-Hodgkin's lymphoma.
(141) The pharmaceutical composition of any one of (135)-(140), wherein the type II anti-CD20 antibody is obinutuzumab.
(142) The pharmaceutical composition of any one of (135)-(141), wherein the caspase activating agent is doxorubicin or a salt thereof.
(143) The pharmaceutical composition of any one of (135)-(142), wherein the obinutuzumab-tolerant CD20-positive cancer is a CD20-positive cancer which was previously treated with obinutuzumab.
(144) The pharmaceutical composition of any one of (135)-(143), wherein the obinutuzumab-tolerant CD20-positive cancer is a cancer which has recurred after initiation of maintenance therapy in which obinutuzumab is administered alone after induction therapy using obinutuzumab.
(145) Use of a type II anti-CD20 antibody in the manufacture of an agent for suppressing cell proliferation of an obinutuzumab-tolerant CD20-positive cancer by combined use with chemotherapy comprising administration of a caspase activating agent.
(146) Use of a caspase activating agent in the manufacture of an agent for suppressing cell proliferation of an obinutuzumab-tolerant CD20-positive cancer by combined use with treatment with a type II anti-CD20 antibody.
(147) Use of a type II anti-CD20 antibody in the manufacture of a pharmaceutical composition for treating an obinutuzumab-tolerant CD20-positive cancer by combined use with chemotherapy comprising administration of a caspase activating agent.
(148) Use of a caspase activating agent in the manufacture of a pharmaceutical composition for treating an obinutuzumab-tolerant CD20-positive cancer by combined use with treatment with a type II anti-CD20 antibody.
(149) Use of a type II anti-CD20 antibody and a caspase activating agent in the manufacture of a pharmaceutical composition for treating an obinutuzumab-tolerant CD20-positive cancer.
(150) The use of any one of (145)-(149), wherein the CD20-positive cancer is B-cell non-Hodgkin's lymphoma.
(151) The use of any one of (145)-(150), wherein the type II anti-CD20 antibody is obinutuzumab.
(152) The use of any one of (145)-(151), wherein the caspase activating agent is doxorubicin or a salt thereof.
(153) The use of any one of (145)-(152), wherein the obinutuzumab-tolerant CD20-positive cancer is a CD20-positive cancer which was previously treated with obinutuzumab.
(154) The use of any one of (145)-(153), wherein the obinutuzumab-tolerant CD20-positive cancer is a cancer which has recurred after initiation of maintenance therapy in which obinutuzumab is administered alone after induction therapy using
(155) A type II anti-CD20 antibody for use in the suppression of cell proliferation of an obinutuzumab-tolerant CD20-positive cancer by combined use with chemotherapy comprising administration of a caspase activating agent.
(156) A caspase activating agent for use in the suppression of cell proliferation of an obinutuzumab-tolerant CD20-positive cancer by combined use with treatment with a type II anti-CD20 antibody.
(157) A type II anti-CD20 antibody for use in the treatment of an obinutuzumab-tolerant CD20-positive cancer by combined use with chemotherapy comprising administration of a caspase activating agent.
(158) A caspase activating agent for use in the treatment of an obinutuzumab-tolerant CD20-positive cancer by combined use with treatment with a type II anti-CD20 antibody.
(159) A combination of a type II anti-CD20 antibody and a caspase activating agent, for use in the treatment of an obinutuzumab-tolerant CD20-positive cancer.
(160) The type II anti-CD20 antibody of (155) or (157), wherein the CD20-positive cancer is B-cell non-Hodgkin's lymphoma.
(161) The caspase activating agent of (156) or (158), wherein the CD20-positive cancer is B-cell non-Hodgkin's lymphoma.
(162) The combination of (159), wherein the CD20-positive cancer is B-cell non-Hodgkin's lymphoma.
(163) The type II anti-CD20 antibody of any one of (155), (157), and (160), wherein the type II anti-CD20 antibody is obinutuzumab.
(164) The caspase activating agent of any one of (156), (158) and (161), wherein the type II anti-CD20 antibody is obinutuzumab.
(165) The combination of (159) or (162), wherein the type II anti-CD20 antibody is obinutuzumab.
(166) The type II anti-CD20 antibody of any one of (155), (157), (160), and (163), wherein the caspase activating agent is doxorubicin or a salt thereof.
(167) The caspase activating agent of any one of (156), (158), (161), and (164), wherein the caspase activating agent is doxorubicin or a salt thereof.
(168) The combination of any one of (159), (162), and (165), wherein the caspase activating agent is doxorubicin or a salt thereof.
(169) The type II anti-CD20 antibody of any one of (155), (157), (160), (163), and (166), wherein the obinutuzumab-tolerant CD20-positive cancer is a CD20-positive cancer which was previously treated with obinutuzumab.
(170) The caspase activating agent of any one of (156), (158), (161), (164), and (167), wherein the obinutuzumab-tolerant CD20-positive cancer is a CD20-positive cancer which was previously treated with obinutuzumab.
(171) The combination of any one of (159), (162), (165), and (168), wherein the obinutuzumab-tolerant CD20-positive cancer is a CD20-positive cancer which was previously treated with obinutuzumab.
(172) The type II anti-CD20 antibody of any one of (155), (157), (160), (163), (166), and (169), wherein the obinutuzumab-tolerant CD20-positive cancer is a cancer which has recurred after initiation of maintenance therapy in which obinutuzumab is administered alone after induction therapy using obinutuzumab.
(173) The caspase activating agent of any one of (156), (158), (161), (164), (167), and (170), wherein the obinutuzumab-tolerant CD20-positive cancer is a cancer which has recurred after initiation of maintenance therapy in which obinutuzumab is administered alone after induction therapy using obinutuzumab.
(174) The combination of any one of (159), (162), (165), (168), and (171), wherein the obinutuzumab-tolerant CD20-positive cancer is a cancer which has recurred after initiation of maintenance therapy in which obinutuzumab is administered alone after induction therapy using obinutuzumab.
(175) A method for suppressing cell proliferation of an obinutuzumab-tolerant CD20-positive cancer, the method comprising:
   i) administering a type II anti-CD20 antibody; and
   ii) administering a caspase activating agent;
   to an obinutuzumab-tolerant CD20-positive cancer cell.
(176) The method of (175), wherein the CD20-positive cancer is B-cell non-Hodgkin's lymphoma.
(177) The method of (175) or (176), wherein the type II anti-CD20 antibody is
(178) The method of any one of (175)-(177), wherein the caspase activating agent is doxorubicin or a salt thereof.
(179) The method of any one of (175)-(178), wherein the obinutuzumab-tolerant CD20-positive cancer is a CD20-positive cancer which was previously treated with obinutuzumab.
(180) The method of any one of (175)-(179), wherein the obinutuzumab-tolerant CD20-positive cancer is a cancer which has recurred after initiation of maintenance therapy in which obinutuzumab is administered alone after induction therapy using obinutuzumab.
(181) The method of any one of (175)-(180), wherein the method is an in vitro method or a non-human in vivo method.
(182) A method for treating an obinutuzumab-tolerant CD20-positive cancer, the method comprising:
   i) administering a type II anti-CD20 antibody; and
   ii) administering a caspase activating agent;
   to an organism having an obinutuzumab-tolerant CD20-positive cancer.
(183) The treatment method of (182), wherein the CD20-positive cancer is B-cell non-Hodgkin's lymphoma.
(184) The treatment method of (182) or (183), wherein the type II anti-CD20 antibody is obinutuzumab.
(185) The treatment method of any one of (182)-(184), wherein the caspase activating agent is doxorubicin or a salt thereof.
(186) The treatment method of any one of (182)-(185), wherein the obinutuzumab-tolerant CD20-positive cancer is a CD20-positive cancer which was previously treated with obinutuzumab.
(187) The treatment method of any one of (182)-(186), wherein the obinutuzumab-tolerant CD20-positive cancer is a cancer which has recurred after initiation of maintenance therapy in which obinutuzumab is administered alone after induction therapy using obinutuzumab.

### EFFECTS OF INVENTION

According to the present invention, the effects of treatments using type II anti-CD20 antibodies, especially obinutuzumab, are increased against CD20-positive cancer that is tolerant to obinutuzumab or CD20-positive cancer that has recurred following an obinutuzumab-containing treatment, by combined use with one or more compounds selected from the group consisting of prednisolone, doxorubicin, vincristine, and salts and prodrugs thereof. Among these compounds, prednisolone, doxorubicin, and salts and prodrugs thereof may be selected. Furthermore, among these compounds, prednisolone or a salt or prodrug thereof may be selected.

### BRIEF DESCRIPTION OF DRAWINGS

FIG. 1 shows the cell proliferation rate, relative to no addition of obinutuzumab, at each concentration of prednisolone added, when obinutuzumab alone or obinutuzumab and prednisolone in combination were allowed to act on obinutuzumab-directed cell death tolerant clone 1A2.

FIG. 2 shows the observation results, obtained by DAPI staining, of the cell cycle ratio upon having allowed obinutuzumab alone, prednisolone alone, or obinutuzumab and prednisolone in combination to act on obinutuzumab-directed cell death tolerant clone 1A2. “Combination” refers to combined use of obinutuzumab and prednisolone.

FIG. 3 shows the observation results, by Western blotting, of the expression levels of intracellular proteins Rb, Skp2, and p27, as well as Rb phosphorylation upon having allowed obinutuzumab alone, prednisolone alone, or obinutuzumab and prednisolone in combination to act on obinutuzumab-directed cell death tolerant clone 1A2.

FIG. 4 shows the observation results, by FACS analysis using TUNEL, of DNA fragmentation upon having allowed obinutuzumab alone, prednisolone alone, or obinutuzumab and prednisolone in combination to act on obinutuzumab-directed cell death tolerant clone 1A2. “Combination” refers to combined use of obinutuzumab and prednisolone.

FIG. 5 shows the cell proliferation rate, relative to no addition of obinutuzumab, at each concentration of doxorubicin added when obinutuzumab alone or obinutuzumab and doxorubicin in combination were allowed to act on obinutuzumab-directed cell death tolerant clone 1A2.

FIG. 6 shows the observation results of caspase 3/7 activity when obinutuzumab alone, doxorubicin alone, or obinutuzumab and doxorubicin in combination were allowed to act on obinutuzumab-directed cell death tolerant clone 1A2, relative to no addition of the two agents.

FIG. 7 shows the observation results, by FACS analysis using TUNEL, of DNA fragmentation upon having allowed obinutuzumab alone, doxorubicin alone, or obinutuzumab and doxorubicin in combination to act on obinutuzumab-directed cell death tolerant clone 1A2, in cases where a pan caspase inhibitor was added and where it was not added. “Combination” refers to combined use of obinutuzumab and doxorubicin.

FIG. 8 shows the cell proliferation rate, relative to no addition of obinutuzumab, when obinutuzumab alone or obinutuzumab and doxorubicin in combination were allowed to act on obinutuzumab-directed cell death tolerant clone 1A2, in the presence or absence of a pan caspase inhibitor.

FIG. 9 shows the ADCC sensitivity when obinutuzumab was allowed to act on a parent RL cell line and ADCC-tolerant cell lines RL-E300-1, RL-E300-2, RL-E300-8, and RL-E300-22.

FIG. 10 shows CD20 expression in the prednisolone-treated group and prednisolone-untreated group of the parent RL cell line and the ADCC-tolerant cell lines RL-E300-1, RL-E300-2, RL-E300-8, and RL-E300-22.

FIG. 11 shows the ADCC sensitivity when obinutuzumab was allowed to act on the parent RL cell line and the ADCC-tolerant cell lines RL-E300-1 and RL-E300-2 after prednisolone treatment, and when obinutuzumab was allowed to act without prednisolone treatment.

FIG. 12 shows the tumor volume in mice grafted with the ADCC-tolerant cell line RL-E300-1 when the mice were administered IgG (30 mg/kg)+vehicle (IgG+Dw group), obinutuzumab (30 mg/kg)+vehicle (OBI+Dw group), IgG (30 mg/kg)+prednisolone (4 mg/kg) (IgG+PSL group), or obinutuzumab (30 mg/kg)+prednisolone (4 mg/kg) (OBI+PSL group).

### DESCRIPTION OF EMBODIMENTS

### A. Agent or medicament for suppressing cell proliferation

### a1) First Embodiment

In one embodiment, the present invention provides an agent or medicament for suppressing cell proliferation of an obinutuzumab-tolerant CD20-positive cancer.

In a first example of the embodiment, the agent comprises a type II anti-CD20 antibody. The agent is used in combination with chemotherapy comprising administration of one or more compounds selected from the group consisting of prednisolone, doxorubicin, vincristine, and salts and prodrugs thereof.

In a second example of the embodiment, the agent comprises one or more compounds selected from the group consisting of prednisolone, doxorubicin, vincristine, and salts and prodrugs thereof. The agent is used in combination with treatment with a type II anti-CD20 antibody.

In a third example of the embodiment, the medicament is a medicament wherein a type II anti-CD20 antibody and one or more compounds selected from the group consisting prednisolone, doxorubicin, vincristine, and salts and prodrugs thereof are concurrently, separately, or sequentially administered in combination.

In the embodiment, B-cell lymphoma is given as an example of CD20-positive cancer. In the embodiment, B-cell non-Hodgkin's lymphoma is given as an example of B-cell lymphoma. In the embodiment, the CD20-positive cancer is preferably B-cell non-Hodgkin's lymphoma.

According to WHO classification, examples of B-cell non-Hodgkin's lymphoma include precursor B lymphoblastic leukemia/lymphoma with precursor B-cell neoplasm being the cell-of-origin; and B-cell chronic lymphocytic leukemia/small lymphocytic lymphoma, B-cell prolymphocytic leukemia, lymphoplasmacytic lymphoma, splenic B-cell marginal zone lymphoma (±villous lymphocytes), hairy cell leukemia, plasma-cell myeloma/plasmacytoma, extranodal marginal zone B-cell lymphoma of MALT type, nodal marginal zone B-cell lymphoma (±monocytoid B cells), follicular lymphoma, mantle cell lymphoma, diffuse large B-cell lymphoma (including mediastinal large B-cell lymphoma and primary effusion lymphoma), and Burkitt's lymphoma with mature B-cell neoplasm being the cell-of-origin. Among the lymphomas in the embodiment, the B-cell non-Hodgkin's lymphoma is preferably follicular lymphoma.

Non-Hodgkin's lymphomas are classified, on the basis of the grade of malignancy thereof, into low-grade lymphomas, intermediate-grade lymphomas, and high-grade lymphomas. Examples of more detailed classification of B-cell non-Hodgkin's lymphomas include Grade 1 or 2 follicular lymphoma and MALT lymphoma as low-grade lymphomas, Grade 3 follicular lymphoma, mantel cell lymphoma, and diffuse large B-cell lymphoma as intermediate-grade lymphomas, and Burkitt's lymphoma as a high-grade lymphoma. On the grade of malignancy basis, the B-cell non-Hodgkin's lymphoma includes follicular lymphoma and is thus preferably a low- or intermediate-grade lymphoma.

In the embodiment, CD20-positive cancer which was previously treated with obinutuzumab is given as an example of an obinutuzumab-tolerant CD20-positive cancer. The term “obinutuzumab tolerance” is interchangeable with “obinutuzumab resistance”. CD20-positive cancer which has recurred after an obinutuzumab-containing treatment was carried out is included in the CD20-positive cancer which was previously treated with obinutuzumab. In the embodiment, the obinutuzumab-tolerant CD20-positive cancer is preferably a CD20-positive cancer which was previously treated with obinutuzumab. The obinutuzumab-tolerant CD20-positive cancer is more preferably B-cell non-Hodgkin's lymphoma which was previously treated with obinutuzumab.

Herein, the term “tolerance” is not limited so long as it refers to a state in which a cell or an individual lacks responsiveness (also referred to as sensitivity) to a treatment or therapy for a disease and/or has a reduced ability to produce a significant response (for example, a partial response and/or a complete response). For example, an obinutuzumab-tolerant cancer is a cancer that either completely lacks responsiveness or does not show a significant response, such as a partial response or a complete response, to a treatment using obinutuzumab. If an agent is administered to a cancer with “tolerance” thereto, not only will desired effects not be obtained, sometimes the cancer may even further progress or turn into a cancer with a higher grade of malignancy. Further, the “tolerance” may be an “intrinsic tolerance or an “acquired tolerance”. In particular, the acquired tolerance in the agents, medicaments etc. of the present invention may be a tolerance that developed after a conventional treatment with obinutuzumab. For example, when the treatment, even if effective in the beginning, continues to be repeated, the cancer may acquire tolerance to the treatment in time, and sometimes the cancer will fail to regress or even progress in the presence of obinutuzumab. Further, as explained in G, “obinutuzumab tolerance” is interchangeable with “anti-CD20 antibody tolerance”. As mentioned below, other than antibodies identified as “Obinutuzumab (Genetical Combination)” in Japan Accepted Name (JAN), obinutuzumab can also include biosimilars and biobetters thereof, as well as type II anti-CD20 antibodies that are antibodies or antigen-binding fragments thereof, which have amino acid sequences with at least 80%, 85%, 90%, 98%, or 99% sequence identity to the amino acid sequence of obinutuzumab. Thus, “obinutuzumab tolerance” can also include tolerance to “Obinutuzumab (Genetical Recombination)”, biosimilars and biobetters, and the above type II anti-CD20 antibodies.

Other examples of obinutuzumab-tolerant CD20-positive cancer include cancer which has recurred after initiation of maintenance therapy in which obinutuzumab is administered alone after induction therapy using obinutuzumab. The induction therapy is a therapy aiming to achieve a response that is at least partial or one in which progression of the disease is not observed, through robust treatment by combined use of obinutuzumab and another chemotherapy. Anti-tumor effects including the partial response or disease progression are assessed on the basis of International Working Group (IWG)'s “Response Criteria on Malignant Lymphoma (Revised)”. The induction therapy is usually continued for 24 weeks. For induction therapy with combined use of obinutuzumab and CHOP therapy or CVP therapy, three weeks of obinutuzumab administration is one cycle and administration is carried out for eight cycles. For induction therapy with combined use with bendamustine, four weeks of obinutuzumab administration is one cycle and administration is carried out for six cycles. In the first cycle of induction therapy, obinutuzumab is administered on days 1, 8, and 15. For the second and subsequent cycles, obinutuzumab is administered on day 1. Where the other chemotherapy is discontinued during induction therapy due to causes such as toxicity, obinutuzumab administration can be continued alone. Meanwhile, the maintenance therapy is a single-agent therapy with obinutuzumab that is continued for a maximum of two years after the induction therapy for patients who have achieved a response that is at least a partial response in the induction therapy. In the maintenance therapy, obinutuzumab is administered once every two months. In the induction therapy and the maintenance therapy, obinutuzumab is administered once a day, at 1000 mg each time. Although the administration method is not particularly limited, administration is preferably by intravenous infusion.

In addition to the above-mentioned cancer which has recurred after initiation of maintenance therapy in which obinutuzumab is administered alone after induction therapy using obinutuzumab, another example of obinutuzumab-tolerant CD20-positive cancer is a CD20-positive cancer which became resistant to obinutuzumab in cases where the other chemotherapy was discontinued and obinutuzumab administration was continued alone in the above-mentioned induction therapy.

In all these embodiments, the carcinoma of the obinutuzumab-resistant CD20-positive cancer is preferably follicular lymphoma.

In the embodiment, the type II anti-CD20 antibody is selected, as appropriate, from those known at the time the agent is manufactured. In the embodiment, obinutuzumab is given as an example of a type II anti-CD20 antibody. In the embodiment, the type II anti-CD20 antibody is preferably obinutuzumab.

The term “anti-CD20 antibody” used herein refers to an antibody which specifically binds to CD20. Based on the binding and biological activity of anti-CD20 antibodies against the CD20 antigen, anti-CD20 antibodies are separated into two types (type I and type II anti-CD20 antibodies) according to Cragg, M. S. et al., Blood 103 (2004) 2738-2743 and Cragg, M. S. et al. Blood 101 (2003) 1045-1052. See Table 1.

TABLE 1
Properties of Type I and Type II Anti-CD20 Antibodies

| | |
|---|---|
| Type I Anti-CD20 Antibodies | Type II Anti-CD20 Antibodies |
| Type I CD20 epitope | Type II CD20 epitope |
| Localize CD20 to lipid rafts | Do not localize CD20 to lipid rafts |
| Increased CDC (if IgG1 isotype) | Reduced CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon | Strong cell death induction without |
| crosslinking | crosslinking |

One essential property of type I and type II anti-CD20 antibodies is the mode of binding thereof. Thus, type land type II anti-CD20 antibodies are classified according to the ratio of the binding capacities to CD20 on Raji cells (ATCC No. CCL-86) of the antibody with respect to rituximab.

Type II anti-CD20 antibodies have a ratio of binding capacities to CD20 on Raji cells (ATCC No. CCL-86), of the anti-CD20 antibody with respect to rituximab, of 0.3-0.6, preferably 0.35-0.55, and more preferably 0.4-0.5. Examples of such type II anti-CD20 antibodies include, e.g., tositumomab (B1 IgG2a), humanized B-Ly1 antibody IgG1 (chimeric humanized IgG1 antibody disclosed in WO 2005/044859 A), 11B8IgG1 (disclosed in WO 2004/035607 A), and AT80IgG1. The type II anti-CD20 antibody is preferably a monoclonal antibody that binds to the same epitope as humanized B-Ly1 antibody (disclosed in WO 2005/044859 A).

In contrast to type II anti-CD20 antibodies, type I anti-CD20 antibodies have a ratio of binding capacities to CD20 on Raji cells (ATCC No. CCL-86), of the anti-CD20 antibody with respect to rituximab, of 0.8-1.2, and preferably 0.9-1.1. Examples of such type I anti-CD20 antibodies include, e.g., rituximab, 1F5IgG2a (ECACC, hybridoma; Press, O. W. et al. Blood 69/2 (1987) 584-591), HI471gG3 (ECACC, hybridoma), 2C6IgG1 (disclosed in WO 2005/103081 A), 2F2IgG1 (disclosed in WO 2004/035607 A and WO 2005/103081 A), and 2H7IgG1 (disclosed in WO 2004/056312 A).

The “ratio of binding capacities to CD20 on Raji cells (ATCC No. CCL-86) of the anti-CD20 antibody with respect to rituximab” is measured through direct immunofluorescence (mean fluorescence intensity (MFI) measured) using the anti-CD20 antibody conjugated to Cy5 and rituximab conjugated to Cy5 in FACS Array (Becton Dickinson) with the Raji cells (ATCC No. CCL-86) described in Example 2, and is calculated as follows.

$$Ratio of binding capacities to CD 20 on Raji Cells ( ATCC No . CCL - 86 ) \frac{M F I ( Cy 5 - anti - CD 20 antibody )}{M F I ( Cy 5 - rituximab )} \times \frac{Cy 5 - labeling ratio ( Cy 5 - rituximab )}{Cy 5 - labeling ratio ( Cy 5 - anti - CD 20 antibody )} \qquad [ Math . 1 ]$$

MFI is the mean fluorescence intensity. The “Cy5 labeling ratio” used herein refers to the number of Cy5 labeling molecules per antibody molecule. Typically, the type II anti-CD20 antibody has a ratio of binding capacity to CD20 on Raji cells (ATCC No. CCL-86), of the type II anti-CD20 antibody with respect to rituximab, of 0.3-0.6, preferably 0.35-0.55, and more preferably 0.4-0.5. The type II anti-CD20 antibodies herein have increased antibody-dependent cellular cytotoxicity (ADCC). An “antibody having increased antibody-dependent cellular cytotoxicity (ADCC)” or an “antibody with increased antibody-dependent cellular cytotoxicity (ADCC)”, as defined herein, has increased ADCC as determined by any suitable method known by those skilled in the art. One of the recognized in vitro ADCC assays is as follows:
1) the above assay uses target cells known to express the target antigen recognized by the antigen-binding region of the antibody;
2) the above assay uses, as effector cells, human peripheral blood mononuclear cells (PBMCs) isolated from the blood of a randomly selected healthy donor;
3) the above assay is carried out according to the following protocol:
   i) PBMCs are isolated using standard density centrifugation and are suspended at 5×10⁶ cells/mL in a RPMI cell culture medium;
   ii) the target cells are grown by standard tissue culture method, harvested from the exponential growth phase with a viability higher than 90%, washed in the RPMI cell culture medium, labeled with 100 μCi of ⁵¹Cr, washed twice with the cell culture medium; and resuspended in the cell culture medium at a density of 10⁵ cells/mL;
   iii) 100 μL of the above final target cell suspension are transferred to each well of a 96-well microtiter plate;
   iv) the antibody is serially diluted from 4000 ng/mL to 0.04 ng/mL in the cell culture medium, 50 μL of the resulting antibody solutions are added to the target cells in the 96-well microtiter plate, and various antibody concentrations covering the entire concentration range above are tested in triplicates;
   v) for maximum release (MR) controls, instead of the antibody solutions (iv above), 50 μL of a 2% (VN) aqueous solution of non-ionic surfactant (Nonidet, Sigma, St. Louis) are added to three additional wells in the plate containing the labeled target cells;
   vi) for spontaneous release (SR) controls, instead of the antibody solutions (iv above), 50 μL of the RPMI cell culture medium are added to three additional wells in the plate containing the labeled target cells;
   vii) the 96-well microtiter plate is then centrifuged at 50×g for one minute and incubated at 4° C. for one hour;
   viii) 50 μL of the PBMC suspension (i above) are added to each well to achieve an effector:target cell ratio of 25:1 and the plate is placed in an incubator under 5% CO₂ atmosphere at 37° C. for four hours:
   ix) the cell-free supernatant from each well is harvested and the experimentally released radioactivity (ER) is quantified using a gamma counter;
   x) the percentage of specific lysis is calculated for each antibody concentration according to the formula (ER−MR)/(MR−SR)×100, where ER is the average radioactivity quantified (see ix above) for that antibody concentration, MR is the average radioactivity quantified (see ix above) for the MR controls (see v above), and SR is the average radioactivity quantified (see ix above) for the SR controls (see vi above);
4) “Increased ADCC” is defined as either an increase in the maximum percentage of specific lysis observed within the antibody concentration range tested above, and/or a reduction in the antibody concentration required to achieve one half of the maximum percentage of specific lysis observed within the antibody concentration range tested above. The increase in ADCC is relative to the ADCC that was measured using the above assay, mediated by the same antibody, produced by the same type of host cells, using the same standard production, purification, formulation and storage methods, which are known to those skilled in the art, but that was not produced by host cells made to overexpress GnTIII.

The “increased ADCC” can be obtained by glycoengineering of the antibodies. This means natural, cell-mediated effector functions of monoclonal antibodies by engineering the oligosaccharide components thereof as described in Umana P. et al. Nature Biotechnol. 17 (1999) 176-180 and U.S. Pat. No. 6,602,684.

The term “complement-dependent cytotoxicity (CDC)” refers to lysis of human tumor target cells by the antibody according to the present invention in the presence of complement. CDC is preferably measured by the treatment of a preparation of CD20-expressing cells with an anti-CD20 antibody according to the present invention in the presence of complement. If the antibody, at a concentration of 100 nM, induces lysis (cell death) of 20% or more of tumor cells after four hours, CDC is observed. This assay preferably uses ⁵¹Cr or Eu-labeled tumor cells and measurement of released ⁵¹Cr or Eu. Controls include incubation of the tumor target cells with complement, and not the antibody.

Typically, type II anti-CD20 antibodies of the IgG1 isotype show characteristic CDC. Type II anti-CD20 antibodies have reduced CDC (if IgG1 isotype) compared with type I anti-CD20 antibodies of the IgG1 isotype. Type II anti-CD20 antibodies are preferably IgG1 isotype antibodies.

The “rituximab” antibody (reference antibody; example of a type I anti-CD20 antibody) is a genetically engineered, chimeric mouse monoclonal antibody directed against the human CD20 antigen, the antibody containing human gamma 1 constant domains . This chimeric antibody is identified by the name “C2B8” in U.S. Pat. No. 5,736,137 (Andersen, K. C. et. al.) of IDEC Pharmaceuticals Corporation, issued on Apr. 17, 1998. Rituximab is approved for the treatment of patients with recurrent, refractory low-grade, follicular, CD20-positive, B cell non-Hodgkin's lymphoma. In vitro mechanism of action studies has shown that rituximab exhibits human complement-dependent cytotoxicity (CDC) (Reff, M. E., et. al., Blood 83 (2) (1994) 435-445). Additionally, it exhibits significant activity in assays that measure antibody-dependent cellular cytotoxicity (ADCC).

The above oligosaccharide component significantly affects properties relevant to the efficacy of a therapeutic glycoprotein, including physical stability, tolerance to protease attack, interactions with the immune system, pharmacokinetics, and specific biological activity. Such properties may depend not only on the presence or absence, but also the specific structures, of oligosaccharides. Some generalization can be made between oligosaccharide structures and glycoprotein functions. For example, certain oligosaccharide structures mediate rapid clearance of the glycoprotein from the bloodstream through interactions with specific carbohydrate binding proteins, while other oligosaccharide structures can be bound by antibodies and trigger undesirable immune reactions. (Jenkins, N., et al., Nature Biotechnol. 14 (1996) 975-81).

Mammalian cells are the preferred hosts for producing therapeutic glycoproteins, due to their capability to glycosylate proteins in the most compatible forms for human application (Cumming, D. A., et al. Glycobiology 1 (1991) 115-30; Jenkins, N., et al., Nature Biotechnol. 14 (1996) 975-81). Bacteria very rarely glycosylate proteins, and like other types of common hosts such as yeasts, filamentous fungi, insect and plant cells, produce glycosylation patterns associated with rapid clearance from the bloodstream, undesirable immune interactions, and in some cases, reduced biological activity. Among mammalian cells, Chinese hamster ovary (CHO) cells have been most commonly used in the last 20 years. In addition to the predetermined suitable glycosylation patterns, these cells allow consistent generation of genetically stable and highly productive clonal cell lines. They are cultured at high concentrations in single bioreactors using serum-free media, allowing the development of safe and reproducible bioprocesses. Other commonly used animal cells include baby hamster kidney (BHK) cells, and NSO- and SP2/0-mouse myeloma cells. More recently, production from transgenic animals has also been tested (Jenkins, N., et al., Nature Biotechnol. 14 (1996) 975-981).

All antibodies contain carbohydrate structures at conserved positions in the heavy chain constant regions, with each isotype possessing a distinct array of N-linked carbohydrate structures, which variably affect protein assembly, secretion, or functional activity (Wright, A. and Morrison, S. L., Trends Biotech. 15 (1997) 26-32). The structure of the attached N-linked carbohydrate varies considerably, depending on the degree of processing, and can include high-mannose, multiply-branched, and biantennary complex oligosaccharides (Wright, A. and Morrison, S. L., Trends Biotech. 15 (1997) 26-32). Typically, there is heterogeneous processing of the core oligosaccharide structures attached at a particular glycosylation site such that even monoclonal antibodies exist as multiple glycoforms. Likewise, it has been shown that major differences in antibody glycosylation occur between cell lines, and even minor differences are seen for a given cell number grown under different culture conditions (Lifely, M. R., et al., Glycobiology 5 (8) (1995) 813-22).

One way to obtain large increases in potency, while maintaining a single production process and potentially avoiding significant, undesirable side effects, is to enhance the natural, cell-mediated effector functions of monoclonal antibodies by engineering their oligosaccharide component as described in Umana, P., et al., Nature Biotechnol. 17 (1999) 176-180 and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody-dependent cellular cytotoxicity (ADCC) (Lifely, M. R., et al., Glycobiology 5 (1995) 813-822; Jefferis, R., et al., Immunol. Rev. 163 (1998) 59-76; Wright, A. and Morrison, S. L., Trends Biotechnol. 15 (1997) 26-32).

It has been shown that overexpression in Chinese hamster ovary (CHO) cells of β(1,4)-N-acetylglucosaminyltransferase III (GnTIII7y), a glycosyltransferase catalyzing the formation of bisected oligosaccharides, significantly increases the in vitro ADCC activity of an anti-neuroblastoma chimeric monoclonal antibody (chCE7) produced by the engineered CHO cells (see Umana, P., et al., Nature Biotechnol. 17 (1999) 176-180 and WO 99/154342 A, the entire contents of which are herein incorporated by reference). The antibody chCE7 belongs to a large class of unconjugated monoclonal antibodies which have high tumor affinity and specificity, but is almost impossible to be clinically useful when produced in standard industrial cell lines lacking the GnTIII enzyme (Umana, P., et al., Nature Biotechnol. 17 (1999) 176-180). That study was the first to show that large increases of ADCC activity could be obtained by engineering the antibody producing cells to express GnTIII, which also led to an increase in the proportion of constant region (Fc)-associated, bisected oligosaccharides, including bisected, non-fucosylated oligosaccharides, to the levels found in naturally-occurring antibodies.

Obinutuzumab, generally a glycoengineered, genetically recombinant humanized anti-CD20 monoclonal antibody, is a glycoprotein that exhibits type II anti-CD20 antibody properties, is composed of two H chains consisting of 449 amino acid residues and two L chains containing 219 amino acid residues, and has a molecular weight of about 148,000-150,000. In the H chain, complementarity-determining region (abbreviated as “CDR”, same hereinafter) 1 is represented by an amino acid sequence consisting of SEQ ID NO: 1, CDR2 is represented by an amino acid sequence consisting of SEQ ID NO: 2, and CDR3 is represented by an amino acid sequence consisting of SEQ ID NO: 3. In the L chain, CDR1 is represented by an amino acid sequence consisting of SEQ ID NO: 4, CDR2 is represented by an amino acid sequence consisting of SEQ ID NO: 5, and CDR3 is represented by an amino acid sequence consisting of SEQ ID NO: 6. The H chain variable region (HV region) is represented by SEQ ID NO: 7. The L chain variable region (LV region) is represented by SEQ ID NO: 8. The H chain is a polypeptide having an amino acid sequence consisting of SEQ ID NO: 9 and the L chain is a polypeptide having an amino acid sequence consisting of SEQ ID NO: 10.

In one embodiment, the type II anti-CD20 antibody may be an antibody or an antigen-binding fragment thereof having an amino acid sequence with at least 80%, 85%, 90%, 98%, or 99% sequence identity to the amino acid sequence of obinutuzumab. In one embodiment, the type II anti-CD20 antibody may be an antibody or an antigen-binding fragment thereof comprising CDRs each comprising an amino acid sequence having at least 80%, 85%, 90%, 95%, 98%, or 99% sequence identity to the amino acid sequence of each CDR in the heavy chain and light chain of obinutuzumab. In one embodiment, the type II anti-CD20 antibody may be an antibody or an antigen-binding fragment thereof comprising a HV region and LV region comprising amino acid sequences with at least 80%, 85%, 90%, 95%, 98%, or 99% sequence identity to the amino acid sequences of the respective HV region and LV region of obinutuzumab.

Obinutuzumab herein includes antibodies identified as “Obinutuzumab (Genetical Recombination)” in Japan Accepted Name (JAN) as well as biosimilars and biobetters thereof. Obinutuzumab is preferably the “Obinutuzumab (Genetical Recombination)” and biosimilars thereof. Herein, biosimilars refer to antibodies having the same amino acid sequences as the above H chain and L chain, optionally with distinct carbohydrate chains from “Obinutuzumab (Genetical Recombination)”, and having biological activity that is equivalent to or higher than that of “Obinutuzumab (Genetical Recombination)”.

Herein, biobetters refer to antibodies having an amino acid sequence similarity of at least 90% and less than 100% to the above H chain and L chain, optionally with distinct carbohydrate chains from “Obinutuzumab (Genetical Recombination)”, and having biological activity that is equivalent to or higher than that of “Obinutuzumab (Genetical Recombination)”.

Type II anti-CD20 antibodies are classified, distinctly from type I, according to their binding and biological activity to the CD20 antigen, and this is described in detail in WO 2009/118142 A.

The type II anti-CD20 antibody, after having been formulated using techniques known at the time of manufacture, is administered to a subject organism. The method for administering the type II anti-CD20 antibody is the same as the embodiment described in “E. Treatment methods” below.

Prednisone is given as an example of a prednisolone prodrug.

Salts of prednisolone, doxorubicin, and vincristine can be selected, as appropriate, from those known to be used in medicaments. An example of a salt of doxorubicin is a hydrochloride thereof. An example of a salt of vincristine is a sulfate thereof.

Prednisolone, doxorubicin, vincristine, and salts thereof, after having been formulated using techniques known at the time of manufacture, are administered to subject organisms. The method for administering these agents is the same as the embodiment described in “E. Treatment methods” below.

In the embodiment, the one or more compounds selected from the group consisting of prednisolone, doxorubicin, vincristine, and salts and prodrugs thereof are preferably selected from the group consisting of prednisolone, doxorubicin, and salts and prodrugs thereof. Furthermore, prednisolone or a salt or prodrug thereof is preferred. The most preferable is prednisolone or prednisone.

### (a2) Second Embodiment

In another embodiment, the “one or more compounds selected from the group consisting of prednisolone, doxorubicin, vincristine, and salts and prodrugs thereof” in the above-mentioned first embodiment may be replaced by a caspase activating agent. Examples of caspase activating agents are described in WO 2004/002428 A and WO 2003/097806 A.

In this other embodiment, caspase 3/7 activating agents are given as examples of caspase activating agents. Doxorubicin or a salt thereof is given as an example of a caspase 3/7 activating agent. In addition to doxorubicin and salts thereof, known caspase 3/7 activating agents may be selected. Examples of known caspase 3/7 activating agents are described in WO 2006/128173 A, WO 2006/074187 A, JP 2008-308455 A, JP 2008-189649 A, and WO 2004/053144 A. The caspase activating agent is preferably doxorubicin or a salt thereof. The other specific configurations of the embodiment are the same as the first embodiment described above.

### (a3) Third Embodiment

In another embodiment, the “agent for suppressing cell proliferation of an obinutuzumab-tolerant CD20-positive cancer” in the above-mentioned first embodiment may be replaced by an enhancing agent of cell cycle arrest or cell death by a type II anti-CD20 antibody in an obinutuzumab-tolerant CD20-positive cancer cell. In the embodiment, the agent comprises prednisolone or a salt or prodrug thereof.

In yet another embodiment, the “agent for suppressing cell proliferation of an obinutuzumab-tolerant CD20-positive cancer” in the above-mentioned first embodiment may be replaced by an inducing agent for enhancing for induction of cell cycle arrest or cell death of an obinutuzumab-tolerant CD20-positive cancer cell. In the embodiment, the agent comprises a type II anti-CD20 antibody.

In yet another embodiment, the “medicament for suppressing cell proliferation of an obinutuzumab-tolerant CD20-positive cancer” in the above-mentioned first embodiment may be replaced by an agent for enhancing induction of cell cycle arrest or cell death against an obinutuzumab-tolerant CD20-positive cancer cell, wherein a type II anti-CD20 antibody and prednisolone or a salt or prodrug thereof are concurrently, separately, or sequentially administered in combination.

In the third embodiment, the cell cycle arrest is not particularly limited and is selected from arrest in any one of the G₀/G₁ phase, S phase, G₂ phase, and M phase. Among these phases, the cell cycle arrest is preferably in the G₀/G₁ phase.

In the third embodiment, where prednisone is used as a prednisolone prodrug, the type II anti-CD20 antibody and prednisone are administered to an organism. In this case, prednisone is converted into prednisolone and acts, together with the type II anti-CD20 antibody, on the obinutuzumab-tolerant CD20-positive cancer in the organism.

In the third embodiment, the prednisolone or a salt or prodrug thereof is preferably prednisolone or prednisone.

The other specific configurations of the third embodiment are the same as the first embodiment described above.

### (a4) Other Configurations in the First to Third Embodiments

In the above first to third embodiments, where the agents are formulated, the formulations are prepared by mixing an antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carrier are generally nontoxic to recipients at the dosages and concentrations employed, and include, but are not limited to, the following: buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose, or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersants such as soluble neutral active hyaluronidase glycoprotein (sHASEGP) (e.g., human soluble PH-20 hyaluronidase glycoproteins such as rHuPH20 (HYLENEX®, Baxter International, Inc.). Specific exemplary sHASEGP and methods of using the same (including rHuPH20) are described in US 2005/0260186 A and US 2006/0104968 A. In one aspect, sHASEGP is combined with one or more additional glycosaminoglycanases such as chondroitinase.

Exemplary lyophilized antibody formulations are described in U.S. Pat. No. 6,267,958. Aqueous solutions of antibody formulations include those described in U.S. Pat. No. 6,171,586 and WO 2006/044908 A, the latter formulation including a histidine-acetate buffer.

The active ingredients may also be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nanoparticles, and nanocapsules), or in macroemulsions. Such techniques are disclosed in Remington's Pharmaceutical Sciences 16th edition, Osol, A. Ed. (1980).

Sustained-release formulations may be prepared. Suitable examples of sustained-release formulations include semipermeable matrices of solid hydrophobic polymers containing the antibody, and the matrices are in the form of shaped articles, e.g., films or microcapsules.

Formulations used for in vivo administration are generally sterile. A sterile state can be easily achieved by, for example, filtering through a sterilizing filter.

When these active ingredients have been separately formulated into two or more formulations, the individual formulations can be concurrently administered, or separately or sequentially administered a set time interval apart. The two or more formulations can also be respectively administered at different frequencies a day. The agents, medicaments, and pharmaceutical compositions according to the present invention can be orally or parenterally administered, systemically or topically. When these active ingredients have been separately formulated into two or more formulations, the individual formulations can also be administered by different routes.

### B. Pharmaceutical Composition

In one embodiment, the present invention provides a pharmaceutical composition for treating an obinutuzumab-tolerant CD20-positive cancer.

In a first example of the embodiment, the pharmaceutical composition comprises a type II anti-CD20 antibody. The pharmaceutical composition is used in combination with chemotherapy comprising administration of one or more compounds selected from the group consisting of prednisolone, doxorubicin, vincristine, and salts and prodrugs thereof.

In a second example of the embodiment, the pharmaceutical composition comprises one or more compounds selected from the group consisting of prednisolone, doxorubicin, vincristine, and salts and prodrugs thereof. The pharmaceutical composition is used in combination with treatment with a type II anti-CD20 antibody.

In the second example of the embodiment, the pharmaceutical composition is a pharmaceutical composition wherein a type II anti-CD20 antibody and one or more compounds selected from the group consisting prednisolone, doxorubicin, vincristine, and salts and prodrugs thereof are concurrently, separately, or sequentially administered in combination.

In the embodiment, general examples and preferred examples of the “type II anti-CD20 antibody”, “one or more compounds selected from the group consisting prednisolone, doxorubicin, vincristine, and salts and prodrugs thereof”, “obinutuzumab-tolerant CD20-positive cancer”, and “formulation” are the same as those described above for the “agent or medicament for suppressing cell proliferation”.

The above-mentioned replacement and the specific configuration of the “one or more compounds selected from the group consisting of prednisolone, doxorubicin, vincristine, and salts and prodrugs thereof” by a “caspase activating agent” mentioned in the second embodiment of the “agent or medicament for suppressing cell proliferation” may also be applied to this embodiment.

The “other configurations in the first to third embodiments” described above may be applied to the other configuration in this embodiment.

### C. Manufacturing Methods

In one embodiment, the present invention provides manufacture of an agent or medicament for suppressing cell proliferation. In the embodiment, the cell is an obinutuzumab-tolerant CD20-positive cancer cell.

In a first example of the embodiment, a type II anti-CD20 antibody is used in the manufacture. The agent is used in combination with chemotherapy comprising administration of one or more compounds selected from the group consisting of prednisolone, doxorubicin, vincristine, and salts and prodrugs thereof.

In a second example of the embodiment, one or more compounds selected from the group consisting of prednisolone, doxorubicin, vincristine, and salts and prodrugs thereof are used in the manufacture. The agent is used in combination with treatment with a type II anti-CD20 antibody.

In a third example of the embodiment, a type II anti-CD20 antibody and one or more compounds selected from the group consisting of prednisolone, doxorubicin, vincristine, and salts and prodrugs thereof are used in the manufacture of the medicament.

In another embodiment, the present invention provides manufacture of a pharmaceutical composition for treating a cancer. In the embodiment, the cancer is an obinutuzumab-tolerant CD20-positive cancer.

In a first example of the embodiment, a type II anti-CD20 antibody is used in the manufacture. The pharmaceutical composition is used in combination with chemotherapy comprising administration of one or more compounds selected from the group consisting of prednisolone, doxorubicin, vincristine, and salts and prodrugs thereof.

In a second example of the embodiment, one or more compounds selected from the group consisting of prednisolone, doxorubicin, vincristine, and salts and prodrugs thereof are used in the manufacture. The pharmaceutical composition is used in combination with treatment with a type II anti-CD20 antibody.

In a third example of the embodiment, a type II anti-CD20 antibody and one or more compounds selected from the group consisting of prednisolone, doxorubicin, vincristine, and salts and prodrugs thereof are used in the manufacture. The type II anti-CD20 antibody and the one or more compounds selected from the group consisting of prednisolone, doxorubicin, vincristine, and salts and prodrugs thereof are concurrently, separately, or sequentially administered in combination.

In these embodiments, obinutuzumab may be manufactured by those skilled in the art according to known methods shown in WO 2005/044859 A. The agents and the pharmaceutical compositions may be manufactured by mixing obinutuzumab with other ingredients using normal techniques.

In these embodiments, general examples and preferred examples of the “type II anti-CD20 antibody”, “one or more compounds selected from the group consisting prednisolone, doxorubicin, vincristine, and salts and prodrugs thereof”, and “obinutuzumab-tolerant CD20-positive cancer” are the same as those described above for the “agent or medicament for suppressing cell proliferation”.

The above-mentioned replacement and specific configuration of the “ one or more compounds selected from the group consisting of prednisolone, doxorubicin, vincristine, and salts and prodrugs thereof” by a “caspase activating agent” mentioned in the second embodiment of the “agent or medicament for suppressing cell proliferation” may also be applied to these embodiments.

Similar to the third embodiment of the “agent or medicament for suppressing cell proliferation” described above, as a first example of the above-mentioned embodiment of “manufacture of an agent for suppressing cell proliferation”, the agent may be replaced by an enhancing agent of cell cycle arrest or cell death by a type II anti-CD20 antibody in an obinutuzumab-tolerant CD20-positive cancer cell. In the manufacture of the embodiment after the replacement, prednisolone or a salt or prodrug thereof is used.

Furthermore, as a second example, the agent may be replaced by an inducing agent of cell cycle arrest or cell death of an obinutuzumab-tolerant CD20-positive cancer cell. In the manufacture of the embodiment after the replacement, a type II anti-CD20 antibody is used. The agent is administered by combined use with chemotherapy comprising administration of prednisolone or a salt or prodrug thereof. With this combined use, cell cycle arrest or cell death against obinutuzumab-tolerant CD20-positive cancer cells is enhanced.

In these examples, the cell cycle arrest is not particularly limited and is selected from arrest in any one of the G₀/G₁ phase, S phase, G₂ phase, and M phase. Among these phases, the cell cycle arrest is preferably in the G₀/G₁ phase.

Furthermore, when prednisone is used as a prednisolone prodrug, the type II anti-CD20 antibody and prednisone are administered to an organism. In this case, prednisone is converted into prednisolone and acts, together with the type II anti-CD20 antibody, on the obinutuzumab-tolerant CD20-positive cancer in the organism.

The other specific configurations in any of these examples are the same as the embodiment of the “manufacture of an agent for suppressing cell proliferation” and the third embodiment of the “agent or medicament for suppressing cell proliferation” described above.

### D. Method for Suppressing Cell Proliferation

In one embodiment, the present invention provides methods for suppressing cell proliferation. In the embodiment, the cell is an obinutuzumab-tolerant CD20-positive cancer cell.

In one embodiment, the method comprises: i) administering a type II anti-CD20 antibody; and ii) administering one or more compounds selected from the group consisting of prednisolone, doxorubicin, vincristine, and salts and prodrugs thereof; to an obinutuzumab-tolerant CD20-positive cancer cell.

The method in the embodiment is an in vitro or in vivo method. The method in the embodiment is preferably an in vitro method or a non-human in vivo method.

In this embodiment, general examples and preferred examples of the “type II anti-CD20 antibody”, “one or more compounds selected from the group consisting prednisolone, doxorubicin, vincristine, and salts and prodrugs thereof”, and “obinutuzumab-tolerant CD20-positive cancer” are the same as those described above for the “agent or medicament for suppressing cell proliferation”.

The above-mentioned replacement of the “one or more compounds selected from the group consisting of prednisolone, doxorubicin, vincristine, and salts and prodrugs thereof” by a “caspase activating agent”, which is an alternative embodiment mentioned in the “agent or medicament for suppressing cell proliferation” above, may also be applied to this embodiment.

Similar to the third embodiment of the “agent or medicament for suppressing cell proliferation” above, as a first example of the above-mentioned embodiment of a “method for suppressing cell proliferation”, the method may be replaced by a method for enhancing cell cycle arrest or cell death by a type II anti-CD20 antibody in an obinutuzumab-tolerant CD20-positive cancer cell. The method of the embodiment after the replacement comprises administration of prednisolone or a salt or prodrug thereof.

Furthermore, as a second example, the method may be replaced by a method for inducing cell cycle arrest or cell death of an obinutuzumab-tolerant CD20-positive cancer cell. The method of the embodiment after the replacement comprises administration of a type II anti-CD20 antibody. This administration is in combination with chemotherapy comprising administration of prednisolone or a salt or prodrug thereof. With this combined use, cell cycle arrest or cell death against obinutuzumab-tolerant CD20-positive cancer cells is enhanced.

In these examples, the cell cycle arrest is not particularly limited and is selected from arrest in any one of the G₀/G₁ phase, S phase, G₂ phase, and M phase. Among these phases, the cell cycle arrest is preferably in the G₀/G₁ phase.

Furthermore, when prednisone is used as a prednisolone prodrug, the type II anti-CD20 antibody and prednisone are administered to an organism. In this case, prednisone is converted into prednisolone and acts, together with the type II anti-CD20 antibody, on the obinutuzumab-tolerant CD20-positive cancer in the organism.

The other specific configurations in any of these examples are the same as the embodiment of the “method for suppressing cell proliferation” and the third embodiment of the “agent or medicament for suppressing cell proliferation” described above.

### E. Treatment Method

In one embodiment, the present invention provides methods for treating CD20-positive cancer. In the embodiment, the CD20-positive cancer is an obinutuzumab-tolerant CD20-positive cancer. The treatment method comprises: i) administering a type II anti-CD20 antibody; and ii) administering one or more compounds selected from the group consisting of prednisolone, doxorubicin, vincristine, and salts and prodrugs thereof; to an organism having an obinutuzumab-tolerant CD20-positive cancer cell.

With respect to administration of the type II anti-CD20 antibody in the embodiment, a therapeutically effective amount thereof may be administered with an administration method known by those skilled in the art. Examples of the administration method include intravenous administration and subcutaneous administration.

With respect to the administration of the one or more compounds selected from the group consisting of prednisolone, doxorubicin, vincristine, and salts and prodrugs thereof in the embodiment, a therapeutically effective amount thereof may be administered with an administration method known by those skilled in the art. Examples of the administration method include intravenous administration, subcutaneous administration, and oral administration.

A “therapeutically effective amount” is the minimal concentration required to at least produce a measurable effect in the improvement or prevention of a specific disorder. The therapeutically effective amount herein may vary depending on factors, e.g., the disease state, age, gender, and weight of the patient, and the antibody's ability to induce a desired response in an individual. A therapeutically effective amount is, additionally, one where therapeutically beneficial effects surpass any toxic or adverse effects of the antibody.

In the treatment method of the present invention, the type II anti-CD20 antibody and the one or more compounds selected from the group consisting of and prednisolone, doxorubicin, vincristine, and salts and prodrugs thereof are preferably administered in combination, and may be administered concurrently, separately, or sequentially.

The type II anti-CD20 antibody and prednisolone, doxorubicin, vincristine, and salts and prodrugs thereof can be concurrently administered, or separately or sequentially administered a set time interval apart, as individual formulations of the agents, medicaments, pharmaceutical compositions, etc. described herein. The two or more formulations may be respectively administered at different frequencies a day and can be respectively administered at different frequencies a certain number of days apart. Each formulation may be administered to one subject at separate dosing schedules as illustrated below.

The type II anti-CD20 antibody, for example, can be administered to an adult human subject by intravenous drip infusion of about 1 mg to about 10 g per administration, preferably about 10 mg to 10 g per administration, preferably about 100 mg to about 10 g per administration, and most preferably about 1000 mg per administration. Administration of the type II anti-CD20 antibody is preferably once daily on the day of administration, but the daily dose can also be divided into two or more doses a day in consideration of the condition of the patient, therapeutic effects, etc.

So long as the desired effects are obtained, the dosage regimen of the type II anti-CD20 antibody is not limited and can be determined, as appropriate, by those skilled in the art, according to the condition of the patient, treatment history, and steps of the treatment (induction therapy or maintenance therapy).

The duration of the treatment using the type II anti-CD20 antibody can continue for a maximum of two years based on the condition of the patient and the therapeutic effects.

Each dosing cycle of the type II anti-CD20 antibody may be about one to about eight weeks, preferably about two to about four weeks, and most preferably about three to about four weeks. The number of cycles for the treatment is not limited so long as the desired effects are obtained, but the treatment may be carried out for about one to about 20 cycles, preferably about three to 10 cycles, and most preferably about five to about eight cycles. Further, during the period in which the type II anti-CD20 antibody is administered in combination with any one of prednisolone, doxorubicin, vincristine, and salts or prodrugs thereof, the type II anti-CD20 antibody is preferably administered for eight cycles, each cycle being about three weeks.

In the dosing cycles constituting the treatment, the type II anti-CD20 antibody may be administered at the same intervals or at different intervals. For example, in the first cycle of the treatment, the antibody may be administered once daily on days 1, 8, and 15 after initiation of the treatment, and once daily on day 1 in the second and subsequent cycles.

In addition, for the type II anti-CD20 antibody, the duration of each cycle and the number of cycles for treatment may be different between induction therapy and maintenance therapy. For example, in induction therapy, about six to about eight cycles may be carried out, each cycle being about three to about four weeks, and in maintenance therapy, administration may continue for two years (about 12 cycles), each cycle being about eight weeks (about two months).

The type II anti-CD20 antibody, for example, may be administered in induction therapy on days 1, 8, and 15 in the first cycle after initiation of the treatment and on day 1 in each of the second and subsequent cycles, each cycle being about three to about four weeks. Thereafter, the treatment in maintenance therapy may continue for a maximum of two years, with each cycle being about eight weeks (about two months).

Prednisone or a salt or prodrug thereof, for example, can be administered to an adult human subject by oral administration at about 1 to about 200 mg per day, based on the condition of the patient and the therapeutic effects, but is preferably administered at about 5 to about 60 mg per day. In addition, although the dose may be given once a day or as two or more divided doses a day, the dose is preferably given once a day or as two to four divided doses a day.

Based on the condition of the patient and the therapeutic effects, doxorubicin or a salt or prodrug thereof, for example, can be administered to an adult human subject by intravenous drip infusion at about 5 to about 100 mg per day, but is preferably administered at about 20 (0.4 mg/kg body weight) to about 30 mg (0.6 mg/kg body weight) per day. In addition, the dose at each administration and the dose per day can be modified, as appropriate, according to the dosage regimen. For example, in the case of intravenous one-shot administration once a day for two to three days followed by about seven to about ten days of withdrawal, it is preferred that about 20 mg be administered once a day and this cycle be repeated for two to three cycles. Alternatively, for example, in the case of intravenous one-shot administration once a day for three consecutive days followed by 18 days of withdrawal, it is preferred that about 20 to about 30 mg be administered once a day and this cycle be repeated for two to three cycles. In addition, doxorubicin or a salt or prodrug thereof, especially when used in combination with another anti-tumor agent, may be administered intravenously once a day at about 25 to about 50 mg/m² (body surface area) (if repeated, the next administration starts with an interval of at least two weeks) or administered intravenously at about 40 mg/m² (body surface area) on day 1, about 30 mg/m² (body surface area) on day 8, followed by 20 days of withdrawal. This cycle may be repeated.

Based on the condition of the patient and the therapeutic effects, vincristine or a salt or prodrug thereof, for example, can be administered to an adult human subject by intravenous drip infusion at about 0.01 to about 0.1 mg/kg body weight per administration, but is preferably administered at about 0.02 to about 0.05 mg/kg body weight per administration. Considering the side effects, it is preferred that each administration not exceed 2 mg. Further, vincristine or a salt or prodrug thereof can be administered with a dosing interval of once in about one to about 14 days, but is preferably administered about once one week.

In the embodiment, the subject of the treatment is an organism having a CD20-positive cancer. The subject is not particularly limited so long as the subject is an organism having a CD20-positive cancer. In the embodiment, mammals including humans are given as examples of the organism. In the embodiment, the organism is preferably a mammal including a human. In the embodiment, rodents and primates including humans are given as examples of the mammals. In the embodiment, mice and rats are given as examples of rodents. In the embodiment, humans and cynomolgus monkeys are given as examples of primates including humans. In the embodiment, the mammal is preferably a primate including a human. In the embodiment, the primate including a human is preferably a human or a cynomolgus monkey, and more preferably a human.

In this embodiment, general examples and preferred examples of the “type II anti-CD20 antibody”, “one or more compounds selected from the group consisting prednisolone, doxorubicin, vincristine, and salts and prodrugs thereof”, and “obinutuzumab-tolerant CD20-positive cancer” are the same as those described above for the “agent or medicament for suppressing cell proliferation”.

The above-mentioned replacement of the “ one or more compounds selected from the group consisting of prednisolone, doxorubicin, vincristine, and salts and prodrugs thereof” by a “caspase activating agent”, which is an alternative embodiment mentioned in the “agent or medicament for suppressing cell proliferation”, may also be applied to this embodiment.

### F. Type II Anti-CD20 Antibody, Compound, and Combination

In one embodiment, the present invention provides type II anti-CD20 antibodies, compounds, and combinations thereof, for use in the suppression of cell proliferation of an obinutuzumab-tolerant CD20-positive cancer, the treatment of an obinutuzumab-tolerant CD20-positive cancer, the enhancement of induction of cell cycle arrest or cell death against an obinutuzumab-tolerant CD20-positive cancer cell, or the enhancement of cell cycle arrest or cell death of an obinutuzumab-tolerant CD20-positive cancer cell.

In a first example of the embodiment, the type II anti-CD20 antibody of the present invention is a type II anti-CD20 antibody for use in combination with chemotherapy comprising administration of one or more compounds selected from the group consisting of prednisolone, doxorubicin, vincristine, and salts and prodrugs thereof.

In a second example of the embodiment, the type II anti-CD20 antibody of the present invention is a type II anti-CD20 antibody for use in combination with chemotherapy comprising administration of a caspase activating agent.

In a third example of the embodiment, the compound of the present invention is one or more compounds selected from the group consisting of prednisolone, doxorubicin, vincristine, and salts and prodrugs thereof, for use in combination with treatment with a type II anti-CD20 antibody.

In a fourth example of the embodiment, the compound is a caspase activating agent for use in combination with treatment with a type II anti-CD20 antibody.

In a fifth example of the embodiment, the combination is a combination of a type II anti-CD20 antibody and one or more compounds selected from the group consisting of prednisolone, doxorubicin, vincristine, and salts and prodrugs thereof.

In a sixth example of the embodiment, the combination is a combination of a type II anti-CD20 antibody and a caspase activating agent.

In one embodiment, the present invention provides prednisolone or a salt or prodrug thereof, for use in the enhancement of cell cycle arrest or cell death of an obinutuzumab-tolerant CD20-positive cancer cell.

In one embodiment, the present invention provides a type II anti-CD20 antibody, and a combination of a type II anti-CD20 antibody and prednisolone or a salt or prodrug thereof, for use in the enhancement of induction of cell cycle arrest or cell death against an obinutuzumab-tolerant CD20-positive cancer cell.

The cell cycle arrest here is not limited so long as cell death is induced, and may be an arrest in the G₀/G₁ phase.

With respect to the type II anti-CD20 antibodies, compounds, and combinations of the present invention, the CD20-positive cancer may be a B-cell non-Hodgkin's lymphoma, the type II anti-CD20 antibody may be obinutuzumab, and the compound may be selected from the group consisting of prednisolone, doxorubicin, and salts and prodrugs thereof. In addition, the obinutuzumab-tolerant CD20-positive cancer may be a CD20-positive cancer which was previously treated with obinutuzumab or a cancer which has recurred after initiation of maintenance therapy in which obinutuzumab is administered alone after induction therapy using obinutuzumab.

With respect to the type II anti-CD20 antibodies, compounds, and combinations of the present invention, general examples and preferred examples of the “type II anti-CD20 antibody”, “one or more compounds selected from the group consisting prednisolone, doxorubicin, vincristine, and salts and prodrugs thereof”, “prednisolone or a salt or prodrug thereof”, and “obinutuzumab-tolerant CD20-positive cancer” are the same as those described above for the “agent or medicament for suppressing cell proliferation”.

The above-mentioned replacement of the “one or more compounds selected from the group consisting of prednisolone, doxorubicin, vincristine, and salts and prodrugs thereof” by a “caspase activating agent”, which is an alternative embodiment mentioned in the “agent or medicament for suppressing cell proliferation”, may also be applied to this embodiment.

### G. Anti-CD20 Antibody-Tolerant CD20-Positive Cancer

In one embodiment, the “obinutuzumab-tolerant CD20-positive cancer” in the above-mentioned embodiments A to F may be replaced by an “anti-CD20 antibody-tolerant CD20-positive cancer”.

The anti-CD20 antibody in the “anti-CD20 antibody-tolerant CD20-positive cancer” includes antibodies that specifically bind to CD20 as defined in A above. The anti-CD20 antibody includes type I and type II anti-CD20 antibodies as defined in Table 1. The anti-CD20 antibody is preferably a type II anti-CD20 antibody. Among type II anti-CD20 antibodies, obinutuzumab is preferred.

Even among CD20-positive cancers, it is a clinically distressing issue as to how to continue using type II anti-CD20 antibodies, such as obinutuzumab, on CD20-positive cancers that are tolerant to obinutuzumab or CD20-positive cancers that have recurred following an obinutuzumab-containing treatment.

As one embodiment under such situations, the present invention found means for increasing the effects of treatments using type II anti-CD20 antibodies, especially obinutuzumab, through combined use with one or more compounds selected from the group consisting of prednisolone, doxorubicin, vincristine, and salts and prodrugs thereof, against CD20-positive cancers that are tolerant to obinutuzumab or CD20-positive cancers that have recurred following an obinutuzumab-containing treatment. Among them, prednisolone, doxorubicin, and salts and prodrugs thereof readily exhibit their effects, and prednisolone or a salt or prodrug thereof tends to exhibit effects particularly readily. Cell cycle arrest in the G₀/G₁ phase through combined use of obinutuzumab and prednisolone is considered as a mechanism involved in the effects.

### INCORPORATION BY REFERENCE

All publications, patents, and patent applications mentioned herein are incorporated herein by reference in their entirety to indicate that each individual publication, patent, or patent application is specifically and individually incorporated by reference. In case of conflict, the present application including definitions herein will prevail.

### EXAMPLES

### Example 1

The human germinal center B-cell-like diffuse large B-cell lymphoma cell line SU-DHL-4 was treated with 100 μg/mL of N-ethyl-N-nitrosourea for 24 hours to randomly introduce gene mutations. Thereafter, obinutuzumab was added to 200 μg/mL, and culturing was carried out at 37° C. in the presence of 5% CO₂ for three weeks. Cells that proliferated were cloned using pico-pipettes under visual inspection with a microscope, and clones 1A2, 1 D2, and 3A4 were established as obinutuzumab-directed cell death-tolerant clones derived from single cells. As a result of a four-day cell proliferation test, the obinutuzumab concentration for 50% cell proliferation inhibition was 0.037 μg/mL in SU-DHL-4 but was at least 200 μg/mL for all three obinutuzumab-directed cell death-tolerant clones, indicating that the obinutuzumab-directed cell death-tolerant clones have reduced sensitivity to obinutuzumab in vitro.

The obinutuzumab-directed cell death-tolerant clones were seeded at 1×10⁴ cells per well in 96-well plates, obinutuzumab (1 μg/mL) was used in combination with an anti-cancer agent selected from 4-hydroperoxycyclophosphamide (100 nM) which is an active metabolite of the alkylating agent cyclophosphamide, doxorubicin (10 nM) which is an anthracycline-based anti-cancer antibiotic, vincristine (1 nM) which is a vinca alkaloid-based anti-cancer agent, or prednisolone (1 μM) which is a synthetic adrenal glucocorticoid. After four days of culturing at 37° C. in the presence of 5% CO₂, the number of viable cells was measured using CellTiter-Glo 3D Cell Viability Assay (Promega). From the proportions of cell proliferation when each agent was added alone and when both agents were added, relative to when no agent was added, the effects of the combined use were quantified with reference to the Bliss independence model, and the results are shown in Table 2. The effects of the combined use were assessed by numerical values obtained as follows: login (proportion of cell proliferation when both agents were added)—log₁₀ (proportion of cell proliferation when obinutuzumab was added alone)—log₁₀ (proportion of cell proliferation when the anti-cancer agent was added alone). Here, for the combined use, a numerical value of less than 0 means a supra-additive effect, 0 means an additive effect, and greater than 0 means a sub-additive effect (J Biopharm Stat. 2012; 22(3): 535-543).

TABLE 2

| Agents Used in Combination | 1A2 | 1D2 | 3A4 |
|---|---|---|---|
| 4-hydroperoxycyclophosphamide | −0.03 | 0.02 | 0.00 |
| doxorubicin | −0.44 | −0.14 | −0.14 |
| vincristine | −0.35 | −0.10 | −0.13 |
| prednisolone | −0.43 | −0.10 | −0.05 |

The results show that in all of the obinutuzumab-directed cell death-tolerant clones, the combined use of obinutuzumab with doxorubicin, prednisolone, or vincristine exhibited supra-additive effects. Meanwhile, the combined use of obinutuzumab with 4-hydroperoxycyclophosphamide exhibited approximately additive effects in all of the obinutuzumab-directed cell death-tolerant clones.

### Example 2

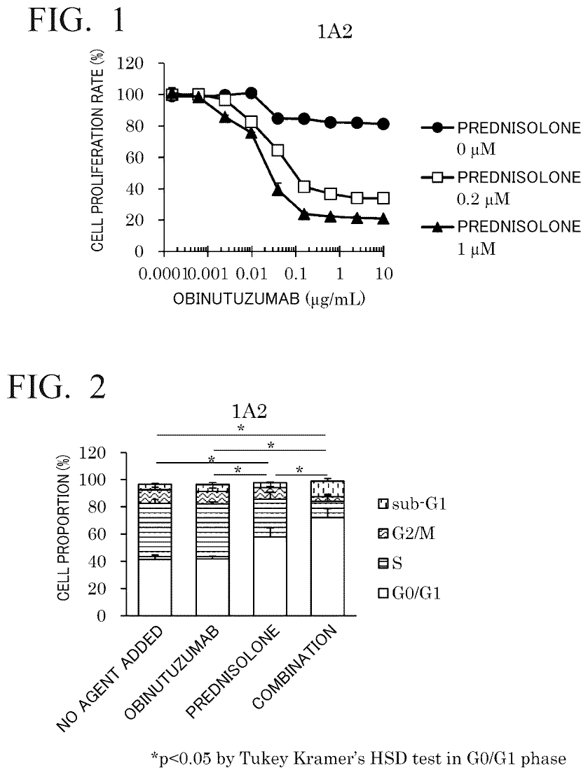

The obinutuzumab-directed cell death-tolerant clone 1A2 was seeded at 1×10⁴ cells per well in 96-well plates, and obinutuzumab (0.00015-10 μg/mL) and prednisolone (0.2, 1 μM) were added. After four days of culturing at 37° C. in the presence of 5% CO₂, the number of viable cells was measured using CellTiter-Glo 3D Cell Viability Assay (Promega). The cell proliferation rate (mean+standard deviation) at each concentration of prednisolone added, relative to the number of cells without obinutuzumab addition, is shown in FIG. 1.

The results show that the combined use with prednisolone enhanced obinutuzumab's inhibitory effect on cell proliferation in the obinutuzumab-directed cell death-tolerant clone.

### Example 3

The obinutuzumab-directed cell death-tolerant clone 1A2 was seeded at 2×10⁵ cells per well in 6-well plates, and obinutuzumab (1 μg/mL) and prednisolone (1 μM) were added. After 48 hours of culturing at 37° C. in the presence of 5% CO₂, the cells were stained with DAPI according to the manufacturer's recommended protocol, and fluorescence was observed using NC-3000 (chemometec). Cell cycle was analyzed using Flow Jo. Version 7.6.5. The results (mean+standard deviation) of three independent tests are shown in FIG. 2. In addition, the “combination” in FIG. 2 indicates the addition of both agents obinutuzumab and prednisolone.

The results show that when both agents, obinutuzumab and prednisolone, were added, there was a statistically significant increase in the proportion of cells in the G₀/G₁ phase compared with when each of the agents was added alone.

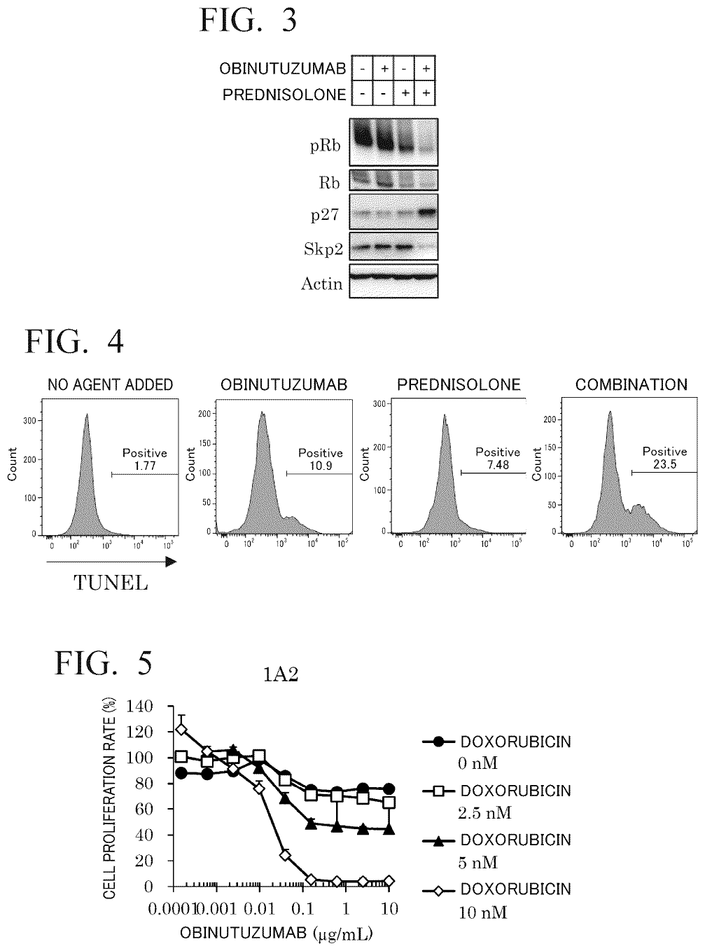

The obinutuzumab-directed cell death-tolerant clone 1A2 was seeded at 3×10⁶ cells per 25 cm² cell culturing flask, and obinutuzumab (1 μg/mL) and prednisolone (1 μM) were added. After 48 hours of culturing at 37° C. in the presence of 5% CO₂, proteins were extracted. Protein expression levels were observed by Western blotting, the results of which are shown in FIG. 3. Further, in the drawing, “−” indicates no addition of agents and “+” indicates an addition of agent(s).

The results show that when obinutuzumab and prednisolone were used in combination, the protein level of the p27-ubiquitin ligase Skp2 decreased, the protein level of the cyclin-dependent kinase inhibitor protein p27 increased, and the level of phosphorylated Rb decreased. Accordingly, the increase in cells in the G₀/G₁ phase with the addition of both agents obinutuzumab and prednisolone, shown in FIG. 2, was believed to be due to a decrease in phosphorylated Rb and an increase in p27, caused by a decrease in Skp2. The foregoing showed a stronger induction of a G₁ phase arrest with the combined use of obinutuzumab and prednisolone, compared with each agent alone.

The obinutuzumab-directed cell death-tolerant clone 1A2 was seeded at 1×10⁶ cells per well in 6-well plates, and obinutuzumab (1 μg/mL) and prednisolone (1 μM) were added. After 72 hours of culturing at 37° C. in the presence of 5% CO₂, cells were stained with APO-BrdU TUNEL Assay Kit (Thermo Fisher). Fluorescence was observed with FACS Fortessa (Becton Dickinson) and the analysis results using Flow Jo. Version 10 are shown in FIG. 4. In addition, the “combination” in FIG. 4 indicates the addition of both agents obinutuzumab and prednisolone.

The results show that the combined use of obinutuzumab and prednisolone promoted DNA fragmentation compared with each agent alone. Thus, the present combined use exhibits a greater enhancement of cell death induction compared with each agent alone.

From the above results, it is believed that obinutuzumab and prednisolone exhibit stronger combined use effects by enhancing G₁ phase arrest and cell death induction.

### Example 4

The obinutuzumab-directed cell death resistant clone 1A2 was seeded at 1×10⁴ cells per well in 96-well plates, and obinutuzumab (0.00015-10 μg/mL) and doxorubicin (2.5-10 nM) were added. After four days of culturing at 37° C. in the presence of 5% CO₂, the number of viable cells was measured using CellTiter-Glo 3D Cell Viability Assay (Promega). The cell proliferation rate (mean+standard deviation) with obinutuzumab addition at each concentration of doxorubicin added, with the cell proliferation rate without obinutuzumab addition being 100%, is shown in FIG. 5.

The results show that the combined use with doxorubicin enhanced obinutuzumab's inhibitory effect on cell proliferation in the obinutuzumab-directed cell death-tolerant clone.

### Example 5

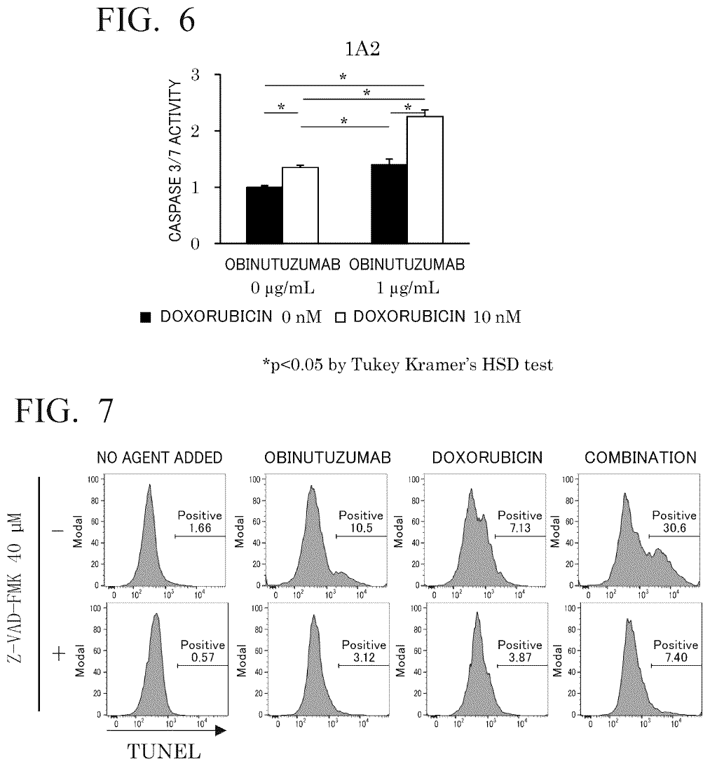

The obinutuzumab-directed cell death resistant clone 1A2 was seeded at 1×10⁴ cells per well in 96-well plates, and obinutuzumab (1 μg/mL) and doxorubicin (10 nM) were added. After 48 hours of culturing at 37° C. in the presence of 5% CO₂, caspase 3/7 activity was measured using CaspaseGlo 3/7 Assay (Promega). The caspase 3/7 activity (mean+standard deviation) relative to cells without addition of the two agents is shown in FIG. 6.

The results show a statistically significant higher caspase 3/7 activity with the combined use of obinutuzumab and doxorubicin compared with each agent alone (P<0.05).

The obinutuzumab-directed cell death resistant clone 1A2 was seeded at 1×10⁶ cells per well in 6-well plates, and obinutuzumab (1 μg/mL), doxorubicin (10 nM), and the pan caspase inhibitor Z-VAD-FMK (40 μM) were added. After 72 hours of culturing at 37° C. in the presence of 5% CO₂, cells were stained with APO-BrdU TUNEL Assay Kit (Thermo Fisher). Fluorescence was observed with FACS Fortessa (Becton Dickinson) and the analysis results using Flow Jo. Version 10 are shown in FIG. 7. In addition, the “combination” in FIG. 7 indicates the addition of both agents obinutuzumab and doxorubicin, the upper panels show the results without Z-VAD-FMK addition, and the lower panels show the results with Z-VAD-FMK addition.

The results show that the combined use of obinutuzumab and doxorubicin promoted DNA fragmentation compared with each agent alone. Thus, the present combined use exhibits a greater enhancement of cell death induction compared with each agent alone. Furthermore, this DNA fragmentation was suppressed by the addition of Z-VAD-FMK. As such, this suggests that the enhanced cell death by the combined use of obinutuzumab and doxorubicin is induced in a caspase-dependent manner.

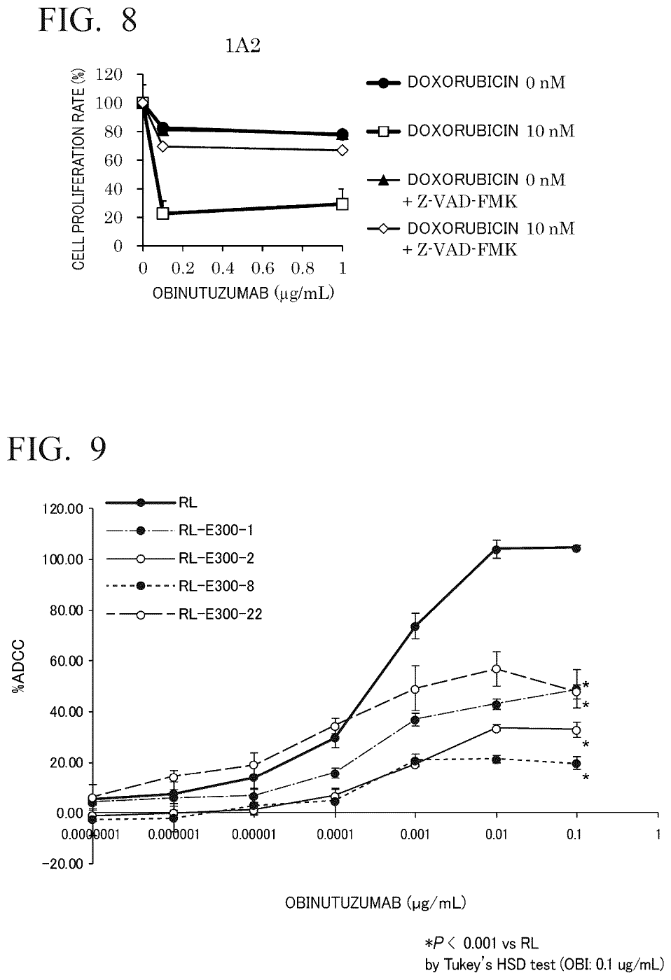

The obinutuzumab-directed cell death resistant clone 1A2 was seeded at 1×10⁴ cells per well in 96-well plates, and obinutuzumab (0.1, 1 μg/mL), doxorubicin (10 nM), and the pan caspase inhibitor Z-VAD-FMK (40 μM) were added. After four days of culturing at 37° C. in the presence of 5% CO₂, the number of viable cells was measured using CellTiter-Glo 3D Cell Viability Assay (Promega). The cell proliferation ratio (mean+standard deviation) with the addition of each agent with respect to the number of cells without obinutuzumab addition is shown in FIG. 8.

The results show that the addition of Z-VAD-FMK suppressed the doxorubicin-enhanced inhibitory activity of obinutuzumab on cell proliferation.

The above results show that obinutuzumab and doxorubicin induce stronger combined use effects by enhancing caspase-dependent cell death.

### Example 6

The human non-Hodgkin's lymphoma cell line RL was treated with 300 μg/mL N-ethyl-N-nitrosourea for 24 hours to randomly introduce gene mutations. CD16(158V)/NK-92 cells were added as effector cells to achieve an effector:target (ET) ratio of 20:1, and ADCC reaction was carried out at 37° C. overnight together with 0.1 μg/mL of obinutuzumab. Cells that proliferated were subjected to a negative selection using an anti-CD56 antibody (Biolegend) and a positive selection using an anti-CD20 antibody (BD Biosciences) by MACS.

After the above ADCC reaction and selections by MACS were repeated a total of three times, the cells that proliferated were cloned to establish obinutuzumab-induced ADCC-tolerant cell lines RL-E300-1, RL-E300-2, RL-E300-8, and RL-E300-22.

The parent RL cell line and the tolerant cell lines were subjected to viable cell staining by Calcein-AM (FUJIFILM Wako Pure Chemical Corporation) and then seeded at 1×10⁴ cells per well in 96-well plates. Obinutuzumab was added at a concentration of 0.0001-100 ng/mL. CD16(158V)/NK-92 cells were added as effector cells to achieve an ET ratio of 1:1 and the cells were incubated at 37° C. for four hours. In addition, for Maximum Lysis, 1% Triton X-100 was added. After the plates were centrifuged and the supernatants were collected, calcein fluorescence was measured using a plate reader. ADCC sensitivity was assessed with the expression (measurement−background)/(Maximum Lysis−background)×100 (%).

The results show that compared with the parent cell line, a significant reduction in ADCC sensitivity was confirmed in all of the ADCC-tolerant cell lines (FIG. 9).

### Example 7

The parent RL cell line and the tolerant cell lines were seeded at 4×10⁵ cells per plate, prednisolone (10 μM) was added, and the cells were cultured at 37° C. for 72 hours. The cells were collected and stained with a control IgG antibody or an anti-CD20 antibody (BD Biosciences), and analysis of CD20 expression was carried out using FACS Fortessa (Becton Dickinson).

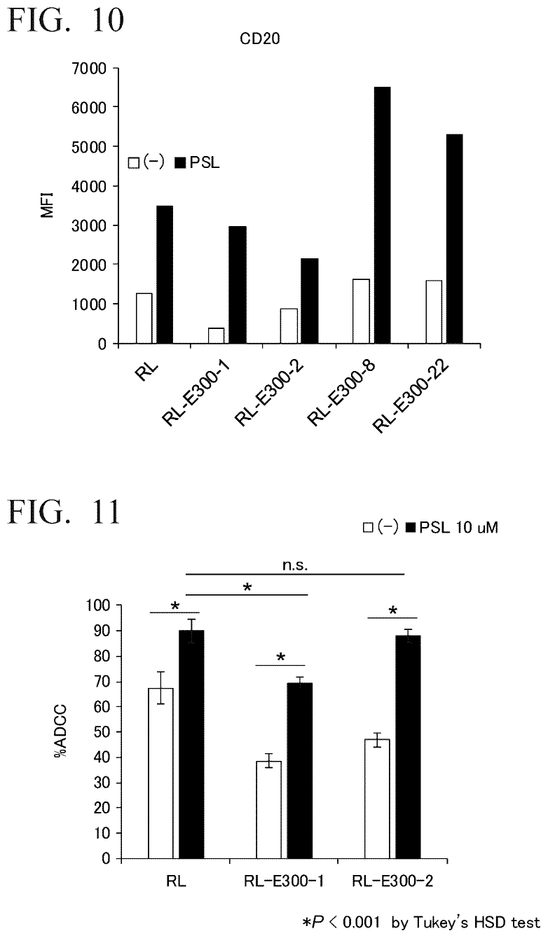

It is clear from the results that the prednisolone (PSL) pre-treatment enhances CD20 expression in the parent cell line and the tolerant cell lines (FIG. 10).

### Example 8

The parent RL cell line and the tolerant cell lines were pre-cultured, with prednisolone (10 μM) added, at 37° C. for 72 hours. The cells were collected and subjected to viable cell staining with Calcein-AM (FUJIFILM Wako Pure Chemical Corporation), and then seeded at 1×10⁴ cells per well in 96-well plates. Obinutuzumab was added at a concentration of 1 ng/mL. CD16(158V)/NK-92 cells were added thereto as effector cells to achieve an ET ratio of 1:1, and the cells were incubated at 37° C. for four hours. In addition, for Maximum Lysis, 1% Triton X-100 was added. After the plates were centrifuged and the supernatants were collected, calcein fluorescence was measured using a plate reader. ADCC sensitivity was assessed with the expression (measurement−background)/(Maximum Lysis−background)×100 (%).

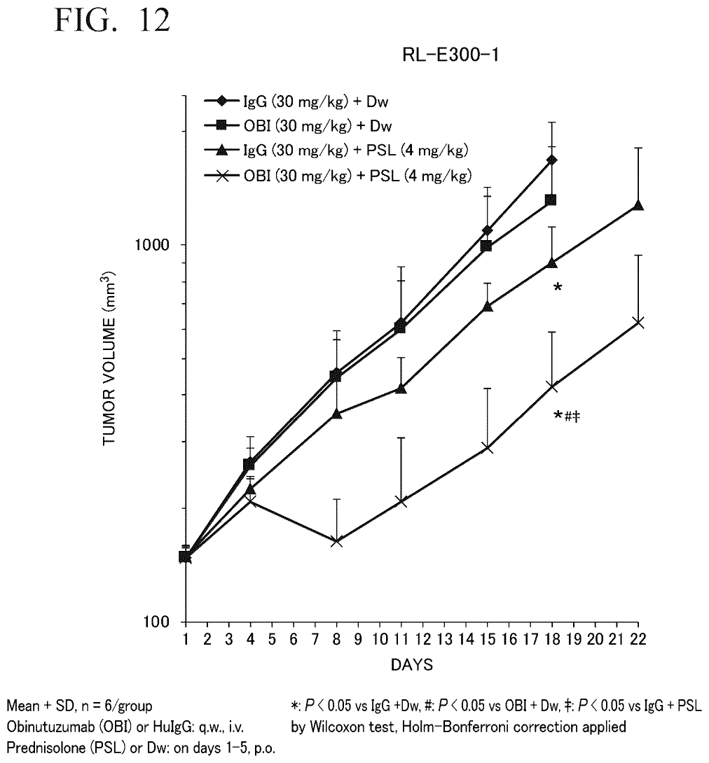

It is clear from the results that the prednisolone (PSL) pre-treatment enhances ADCC sensitivity in the parent cell line and the tolerant cell lines (FIG. 11).

### Example 9

The tolerant cell line RL-E300-1 was subcutaneously grafted into C.B-17/Icr-scid/scidJcl mice (CLEA Japan) at 5×10⁶ cells/mouse, and after tumor engraftment, the mice were divided into the following groups (n=6 in each group).
1. IgG (30 mg/kg)+vehicle administration group (IgG+Dw group)
2. Obinutuzumab (30 mg/kg)+vehicle administration group (OBI+Dw group)
3. IgG (30 mg/kg)+prednisolone (4 mg/kg) administration group (IgG+PSL group)
4. Obinutuzumab (30 mg/kg)+prednisolone (4 mg/kg) administration group (OBI+PSL group)

Administration was performed (obinutuzumab or IgG (human IgG, CAPPEL, Cat#55908): days 1, 8, 15 (once a week (q.w.), intravenous infusion (i.v.); prednisolone or vehicle (Dw): days 1-5, oral administration (p.o.)) and tumor size was measured.

The results show that when the groups were compared on day 18, a significant anti-tumor effect was confirmed in the combination group (OBI+PSL group) compared with each control group (IgG+Dw, OBI+Dw, or IgG+PSL group) (FIG. 12).

1. A method of treating a subject having an obinutuzumab-tolerant CD20-positive cancer by administering to the subject:
(i) a type II anti-CD20 antibody; and
(ii) one or more compounds selected from the group consisting of prednisolone, doxorubicin, vincristine, and salts and prodrugs thereof.

2. (canceled)

3. The method of claim 1, wherein the obinutuzumab-tolerant CD20-positive cancer is B-cell non-Hodgkin's lymphoma.

4. The method of claim 1, wherein the type II anti-CD20 antibody is obinutuzumab.

5. The method of claim 1, wherein the one or more compounds are selected from the group consisting of prednisolone, doxorubicin, and salts and prodrugs thereof.

6. The method of claim 1, wherein the obinutuzumab-tolerant CD20-positive cancer is a CD20-positive cancer which was previously treated with obinutuzumab.

7. The method of claim 1, wherein the obinutuzumab-tolerant CD20-positive cancer is a cancer which has recurred after initiation of maintenance therapy in which obinutuzumab is administered alone after induction therapy using obinutuzumab.

8-14. (canceled)

15. The method of claim 1, wherein the type II anti-CD20 antibody is administered in a pharmaceutical composition.

16. A method of suppressing cell proliferation of an obinutuzumab-tolerant CD20-positive cancer in a subject by administering to the subject:
(i) a type II anti-CD20 antibody; and
(ii) one or more compounds selected from the group consisting of prednisolone, doxorubicin, vincristine, and salts and prodrugs thereof.

17. The method of claim 16, wherein the obinutuzumab-tolerant CD20-positive cancer is B-cell non-Hodgkin's lymphoma.

18. The method of claim 16, wherein the type II anti-CD20 antibody is obinutuzumab.

19. The method of claim 16, wherein the one or more compounds are selected from the group consisting of prednisolone, doxorubicin, and salts and prodrugs thereof.

20. The method of claim 16, wherein the obinutuzumab-tolerant CD20-positive cancer is a CD20-positive cancer which was previously treated with obinutuzumab.

21. The method of claim 16, wherein the obinutuzumab-tolerant CD20-positive cancer is a cancer which has recurred after initiation of maintenance therapy in which obinutuzumab is administered alone after induction therapy using obinutuzumab.

22. The method of claim 16, wherein the type II anti-CD20 antibody is administered in a pharmaceutical composition.

23. A method of enhancing cell cycle arrest or cell death in an obinutuzumab-tolerant CD20-positive cancer cell, the method comprising administering (i) a type II anti-CD20 antibody and (ii) prednisolone or a salt or prodrug thereof to the cancer cell.

24. The method of claim 23, wherein the prodrug is prednisone, and wherein the type II anti-CD20 antibody and the prednisone are administered to an organism.

25. The method of claim 23, wherein the type II anti-CD20 antibody is obinutuzumab.

26. The method of claim 23, wherein the cell cycle arrest is an arrest in G0/G1 phase.

27. The method of claim 23, wherein the obinutuzumab-tolerant CD20-positive cancer is B-cell non-Hodgkin's lymphoma.

28. The method of claim 23, wherein the obinutuzumab-tolerant CD20-positive cancer is:
(i) a CD20-positive cancer which was previously treated with obinutuzumab; or
(ii) a cancer which has recurred after initiation of maintenance therapy in which obinutuzumab is administered alone after induction therapy using obinutuzumab.

29. The method of claim 23, wherein the type II anti-CD20 antibody is administered in a pharmaceutical composition.


SEQUENCE LISTING (10 sequences)

SEQ ID NO: 1 - protein, 6 aa, Artificial Sequence
  H-CDR1
GYAFSY

SEQ ID NO: 2 - protein, 8 aa, Artificial Sequence
  H-CDR2
FPGDGDTD

SEQ ID NO: 3 - protein, 10 aa, Artificial Sequence
  H-CDR3
NVFDGYWLVY

SEQ ID NO: 4 - protein, 16 aa, Artificial Sequence
  L-CDR1
RSSKSLLHSNGITYLY

SEQ ID NO: 5 - protein, 7 aa, Artificial Sequence
  L-CDR2
QMSNLVS

SEQ ID NO: 6 - protein, 9 aa, Artificial Sequence
  L-CDR3
AQNLELPYT

SEQ ID NO: 7 - protein, 119 aa, Artificial Sequence
  H-variable region
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 115 aa, Artificial Sequence
  L-variable region
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 9 - protein, 449 aa, Artificial Sequence
  full length H chain
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Artificial Sequence
  full length L chain
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC